(19) United States
(12) Patent Application Publication
(10) Pub. No.: US 2022/0331433 A1
(43) Pub. Date: Oct. 20, 2022

(54) NUCLEIC ACID-MEDIATED DELIVERY OF THERAPEUTICS

(71) Applicants: The Regents of the University of California, Oakland, CA (US); PHARMARESEARCH CO., LTD., Gangneung-si (KR)

(72) Inventors: Young Jik Kwon, Irvine, CA (US); Saad Noor Mohammad, Irvine, CA (US); Iksoo Kim, Gangneung-si (KR); Seunggul Baek, Gangneung-si (KR)

(21) Appl. No.: 17/640,758

(22) PCT Filed: Sep. 4, 2020
(86) PCT No.: PCT/US2020/049580

§ 371 Date: Mar. 4, 2022

Related U.S. Application Data
(60) Provisional application No. 62/897,254, filed on Sep. 6, 2019.

(51) Int. Cl. A61K 47/54; A61K 31/7048; A61K 9/51; A61K 31/704

(52) U.S. Cl. CPC A61K 47/549; A61K 31/7048; A61K 9/51; A61K 31/704

(57) ABSTRACT

The disclosure provides for compositions comprising one or more therapeutic compounds that are complexed with nucleic acid fragments to form nanoparticles, and uses thereof.

### CROSS REFERENCE TO RELATED APPLICATIONS

This application claims priority under 35 U.S.C. § 119 from Provisional Application Ser. No. 62/897,254 filed Sep. 6, 2019, the disclosure of which is incorporated herein by reference.

### TECHNICAL FIELD

The disclosure provides for nucleic acid-mediated delivery of therapeutics that can associate or bind with DNA or RNA, and uses thereof.

### BACKGROUND

Therapeutics that can associate or bind with DNA or RNA have great potential for treating cancers and other diseases, but their inherent chemical structure can make them fully or partially insoluble leading to limited bioavailability. Some of these therapeutics, while soluble, can lead to systemic toxicity, and can often be cleared too quickly from the body.

### SUMMARY

Provided herein is a delivery platform for therapeutics that can associate or bind with DNA or RNA, which is more efficacious than current formulations, and that has further advantages in cost of production and ease of assembly. In the exemplary studies presented herein a mixture of nucleic acid fragments ranging from 50 to 2,000 nucleotides were used as a bioactive nanocarrier for doxorubicin (DOX), an intercalating agent. It was found that DOX could be complexed with the nucleic acid fragments in a rapid and facile manner. This DOX/nucleic acid formulation was much more monodispersed than the nucleic acid fragments themselves and improved the therapeutic window of DOX. As indicated in the studies herein, it is clear that the delivery of therapeutics that can associate or bind with DNA or RNA in general can be improved by use of the delivery platform disclosed herein.

In a particular embodiment, the disclosure provides for composition comprising one or more therapeutic compounds that are complexed with nucleic acid fragments to form nanoparticles. In another embodiment or a further embodiment of any of the foregoing embodiments, the one or more therapeutic compounds are small molecules that can associate or bind with DNA or RNA. In another embodiment or a further embodiment of any of the foregoing embodiments, the nucleic acid fragments are complexed with the one or more therapeutic compounds at a wt/wt ratio of 2:1 to 10:1. In another embodiment or a further embodiment of any of the foregoing embodiments, the nucleic acid fragments are complexed with the one or more therapeutic compounds at a wt/wt ratio of 4:1 to 7:1. In another embodiment or a further embodiment of any of the foregoing embodiments, the nucleic acid fragments are complexed with the one or more therapeutic compounds at a wt/wt ratio of about 6:1. In another embodiment or a further embodiment of any of the foregoing embodiments, the nanoparticles are from 20 nm to 200 nm in size. In another embodiment or a further embodiment of any of the foregoing embodiments, the nanoparticles are from 50 nm to 100 nm in size. In another embodiment or a further embodiment of any of the foregoing embodiments, the one or more therapeutic compounds comprises anthracyclines, anthracenediones, camptotheca compounds, podophyllum compounds, minor groove binders, bleomycin, and/or actinomycin D. In another embodiment or a further embodiment of any of the foregoing embodiments, the one or more therapeutic compounds comprises aclarubicin, doxorubicin, daunorubicin, idarubicin, epirubicin, amrubicin, pirarubicin, valrubicin, and/or zorubicin. In another embodiment or a further embodiment of any of the foregoing embodiments, the one or more therapeutic compounds comprises doxorubicin. In another embodiment or a further embodiment of any of the foregoing embodiments, the one or more therapeutic compounds comprises mitoxantrone, topetecan, etoposide, teniposide, bleomycin, actinomycin D, and/or duocarmycin A. In another embodiment or a further embodiment of any of the foregoing embodiments, the one or more of the nucleic acid fragments comprise a ligand that targets the nanoparticles to specific cells, tissue, organs, or tumors. In another embodiment or a further embodiment of any of the foregoing embodiments, the nucleic acid fragments comprise fragments of naturally occurring DNA, RNA and/or DNA-RNA hybrids. In another embodiment or a further embodiment of any of the foregoing embodiments, the nucleic acid fragments comprise chemically synthesized DNA, RNA and/or DNA-RNA hybrids of differing nucleotide lengths. In another embodiment or a further embodiment of any of the foregoing embodiments, the RNA has been modified to replace the 2′ ribose hydroxyl group with an —O-alkyl group or a halide. In another embodiment or a further embodiment of any of the foregoing embodiments, the nucleic acid fragments are DNA fragments. In another embodiment or a further embodiment of any of the foregoing embodiments, the DNA fragments are from salmon DNA. In another embodiment or a further embodiment of any of the foregoing embodiments, the nucleic acid fragments are from 20 nt to 10,000 nt in length. In another embodiment or a further embodiment of any of the foregoing embodiments, the nucleic acid fragments are from 50 nt to 2,000 nt in length. In another embodiment or a further embodiment of any of the foregoing embodiments, the composition comprises nanoparticles of one or more therapeutic compounds complexed with DNA fragments from 50 nt to 2,000 nt in length. In another embodiment or a further embodiment of any of the foregoing embodiments, the one or more therapeutic compounds is selected from aclarubicin, doxorubicin, daunorubicin, idarubicin, epirubicin, amrubicin, pirarubicin, valrubicin, and zorubicin. In another embodiment or a further embodiment of any of the foregoing embodiments, the one or more therapeutic compounds is doxorubicin.

In a certain embodiment, the disclosure also provides a pharmaceutical composition comprising a composition disclosed herein and a pharmaceutically acceptable carrier, diluent, and/or excipient. In a further embodiment, the pharmaceutical composition is formulated for parenteral delivery.

In a particular embodiment, the disclosure further provides a method of treating a subject having a cancer in need of treatment thereof, comprising: administering to the subject an effective amount of a pharmaceutical composition disclosed herein. In a further embodiment, the cancer is selected from acute lymphoblastic leukemia, acute myeloblastic leukemia, bone sarcoma, breast cancer, endometrial cancer, gastric cancer, head and neck cancer, Hodgkin lymphoma, Non-Hodgkin lymphoma, liver cancer, kidney cancer, multiple myeloma, neuroblastoma, ovarian cancer, small cell lung cancer, soft tissue sarcoma, thyomas, thyroid cancer, transitional cell bladder cancer, uterine sarcoma, Wilms' tumor, and Waldenström macroglobulinemia.

In a certain embodiment, the disclosure provides a human subject having a cancer in need of treatment thereof, comprising: administering an effective amount of a composition disclosed herein. In a further embodiment, the cancer is selected from acute lymphoblastic leukemia, acute myeloblastic leukemia, bone sarcoma, breast cancer, endometrial cancer, gastric cancer, head and neck cancer, Hodgkin lymphoma, Non-Hodgkin lymphoma, liver cancer, kidney cancer, multiple myeloma, neuroblastoma, ovarian cancer, small cell lung cancer, soft tissue sarcoma, thyomas, thyroid cancer, transitional cell bladder cancer, uterine sarcoma, Wilms' tumor, and Waldenström macroglobulinemia. In another embodiment or a further embodiment of any of the foregoing embodiments, the method further comprises administering to the subject with one or more anticancer agents selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors. In another embodiment or a further embodiment of any of the foregoing embodiments, the method further comprises administering to the subject with one or more anticancer agents selected from mitoxantrone, topetecan, etoposide, teniposide, bleomycin, actinomycin D, and duocarmycin A.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 shows that DNA quenches DOX fluorescence at a 6:1 ratio (w/w). Fluorescence spectra of DNA:DOX ratios between 1-100 were first measured (top spectra), then fluorescence spectra of DNA:DOX ratios between 1-10 was measured (bottom spectra). Excitation was carried out at 490 nm. The loading capacity and encapsulation efficiency were determined to be ˜14% and ˜88%, respectively. The weight ratio determined from the studies was found to be 6:1 DNA to DOX.

FIG. 2 presents transmission electron microscopy (TEM) images of DNA (top image) or DOX/DNA (bottom image) prepared in water. DNA was added to DOX at a 6:1 w/w ratio. Time was allotted for self-assembly. In this specific case, water was added to the mixture, and the solution was allowed to rest for a further 30 minutes. Final [DNA]=6 μg/mL and final [DOX]=1 μg/mL.

FIG. 3 presents TEM images of DNA (top image) and DOX/DNA nanoparticles (bottom image). DOX/DNA and DNA were both prepared in PBS before diluting in H₂O for imaging. Final [DNA]=6 μg/mL and final [DOX]=1 μg/mL. The DOX/DNA nanoparticles were approximately 70 nm in size.

FIG. 4 presents TEM images of DOX/DNA reconstituted from lyophilization. Lower magnification (left panel) and higher magnification (right panel) of DOX/DNA are shown. DOX/DNA was prepared in PBS, diluted with H₂O, lyophilized overnight, then reconstituted with H₂O and subsequently imaged. Final [DNA]=6 μg/mL and final [DOX]=1 μg/mL.

FIG. 5 presents another set of TEM images of DOX/DNA reconstituted from lyophilization. Lower magnification (left panel) and higher magnification (right panel) of DOX/DNA are shown. Final [DNA]=6 μg/mL and final [DOX]=1 μg/mL.

FIG. 6 presents a further set of TEM images of DOX/DNA reconstituted from lyophilization. High magnification (left panel) and very high magnification (right panel) of DOX/DNA are shown. Final [DNA]=6 μg/mL and final [DOX]=1 μg/mL.

FIG. 7 presents two TEM images of DNA prepared in PBS and diluted in H₂O. Final [DNA]=6 μg/mL.

FIG. 8 presents two additional TEM images at a lower magnification of DNA prepared in PBS and diluted in H₂O. Final [DNA]=6 μg/mL.

FIG. 9 presents a gel photo of a DNA degradation assay in 10% FBS/PBS. DNA was incubated with serum-containing PBS at 37° C. over time. Samples were stored in −20° C. to stop enzymatic degradation from nucleases at each time point. [DNA]=100 μg/mL. Numbers on left indicate the base pairs in the ladder (L). 0*: fresh DNA (no −20° C. storage). DNA is degraded overtime when exposed to 10% FBS/PBS. It is likely that the nucleases in the serum-containing media are contributing to this degradation. It is possible to infer a delayed release of DOX due to this degradation of DNA overtime in 10% FBS.

FIG. 10 provides the results of a study looking at in vitro cytotoxicity of DOX/DNA in EL4 cells at 24 h, 48 h and at 72 h. EL4 cells were treated in triplicate for 24, 48, and 72 hours with a range of concentrations, followed by cellular viability analysis via an MTT assay. DOX/DNA exhibited less cytotoxicity on these cells than DOX by a ˜3.5-fold difference at 24 h of incubation, as seen in the reported IC₅₀ values: DOX/DNA IC₅₀=1.143 μg/mL or 2.1 μM and DOX IC₅₀=0.313 μg/mL or 0.576 μM. DOX/DNA exhibited similar cytotoxicity on these cells compared to DOX at 48 and 72 h of incubation, as seen in the reported IC₅₀ values: DOX/DNA IC₅₀=0.072 μg/mL and DOX IC₅₀=0.093 μg/mL at 48 h or DOX/DNA IC₅₀=0.055 μg/mL and DOX IC₅₀=0.048 μg/mL at 72 h. This result in tandem with the 24 h cytotoxicity data suggests a delayed release of DOX from DOX/DNA. Moreover, the results demonstrate that the nanoparticles exhibit less toxicity in comparison to their free small molecule counterpart.

FIG. 11 presents the results of pharmacokinetics study performed on EL4-challenged C57BL/6 mice treated i.v with either 20 mg/kg DOX or 20 mg/kg DOX equiv. of DOX/DNA. The mice were 6-8-week-old female mice. It was found that DOX/DNA has a longer blood circulation residence half-life (DOX/DNA: T_{1/2}=75 minutes) in EL4-challenged C57BL/6 mice compared to mice treated with DOX (DOX: T_{1/2}=3 minutes), n=3. DOX is absorbed by the tissue in ˜15 minutes (as indicated by the steep initial slope of the curve), then a profile more reminiscent of hepatic and renal clearance was seen. DOX/DNA, however, exhibits a far less steep tissue absorption profile that endures for 1 hour. It can be inferred from the foregoing results, that there was enhanced circulation of DOX and DOX protection/shielding due to DNA. After which, a profile indicative of hepatic and renal clearance was observed. Accordingly, the drug delivery system of the disclosure alters the dissolution and absorption of doxorubicin, possibly allowing for sustained release of the active agent.

FIG. 12 provides binding kinetics of DOX with DNA. Fluorescence of DOX/DNA was measured as [DOX] was increased. [DNA] remained constant at 400 μg/mL, n=3.

FIG. 13 demonstrates that DOX binding to DNA decreases over 24 h in FBS and serum-containing PBS. [DNA] is constant at 400 μg/mL. It is likely that DOX is released from DNA due to FBS.

FIG. 14 demonstrates that the serum content in the media resulted in DOX being released from DOX/DNA over time in a dose dependent manner, repeated experiments of n=3. This data in tandem with the binding kinetics experiment suggests that DOX is released from DOX/DNA over time depending on the amount of serum in the media. The majority of DOX should be released from the nanoparticle over 72 hours, at least according to this model.

FIG. 15 provides a complete blood count and liver enzyme panel conducted in C57BL/6 mice treated with 20 mg/kg DOX, 20 mg/kg DOX equiv. of DOX/DNA, PBS, or 120 mg/kg DNA, n=3.

FIG. 16 provides for the biodistribution of DOX vs DOX/DNA at multiple time points after i.v injection. DOX accumulated in organs and tumor tissue after a 20 mg/kg i.v. administration of DOX, DOXIL (20 mg/kg DOX equivalent), or DOX/DNA (20 mg/kg DOX equivalent) in EL4-challenged C57BL/6 mice (Female, 6-8 weeks old) at 1, 3, 6, and 12 hours. Accumulation of DOX in lungs is lower in DOX/DNA group, n=5 (except for DOXIL 12 h, where n=3).

FIG. 17 provides for the biodistribution of DOX vs DOX/DNA at multiple time points after i.v injection in EL4-challenged C57BL/6 mice. DOX accumulated in organs and tumor after a 20 mg/kg i.v. administration of DOX, DOXIL (20 mg/kg DOX equivalent), or DOX/DNA (20 mg/kg DOX equivalent) in EL4-challenged C57BL/6 mice (Female, 6-8 weeks old) at 1, 3, 6, and 12 hours. Accumulation of DOX in lungs is lower in DOX/DNA group, n=5 (except for DOXIL 12 h, where n=3).

FIG. 18 provides for the biodistribution of DOX vs DOX/DNA at multiple time points after i.v. injection in EL4-challenged C57BL/6 mice. DOX accumulated in organs and tumor after a 20 mg/kg i.v. administration of DOX, DOXIL (20 mg/kg DOX equivalent), or DOX/DNA (20 mg/kg DOX equivalent) in EL4-challenged C57BL/6 mice (Female, 6-8 weeks old) at 1, 3, 6, and 12 hours. Accumulation of DOX in lungs is lower in DOX/DNA group, n=5 (except for DOXIL 12 h, where n=3).

FIG. 19 provides acute toxicity survival curves in C57BL/6 mice (Female, 6-8 weeks), n=7. Acute toxicity was not observed in dose regimens 20 mg/kg or below. DOX-treated mice experienced acute toxicity (cardiac arrest) due to 40 mg/kg dose administration.

FIG. 20 shows tumor growth and survival of EL4-challenged mice that were tracked regularly for 30 days after i.v. treatment with DOX/DNA, DOX, or DOXIL in a range of doses (2-3-month-old female mice). DOX/DNA slowed tumor growth and improved the survival rate in EL4-challenged C57BL/6 mice, n=5. The 20 mg/kg dosage exhibited prolonged survival and slowed tumor growth when using the nanocarrier formulation. Interestingly, complete tumor regression was observed until day 28 in mice treated with 40 mg/kg of DOX/DNA. Moreover, 60% of these mice survived to the endpoint of the experiment. These results effectively demonstrate DNA's ability to increase the maximum tolerated dose of DOX, in addition to demonstrating its protective effects against systemic toxicity. This could possibly translate to improved survival and improved quality of life in human treated subjects. The weight of EL4-challenged mice was tracked regularly for 30 days after i.v. treatment of DOX/DNA, DOX, or DOXIL with a range of doses (2-3-month-old female mice). DOX/DNA treatment lead to weight loss in high dose treatment in EL4-challenged C57BL/6 mice, n=5.

FIG. 21 provides weight, tumor growth, and survival outcomes for EL4-challenged C57BL/6 mice (6 weeks) treated with 20 mg/kg DOX or DOX equiv. of DOXIL or DOX/DNA on day 0, day 7, and day 14, n=5. DOX/DNA treatment conferred a more promising outcome compared to free DOX or DOXIL. DOX/DNA is a safer alternative than free DOX. Further, the DOX/DNA treatment had the most pronounced reduction in tumor growth, and had the best survival outcome.

FIG. 22 demonstrates that DOX/DNA uptake in EL4 cells was inhibited by endocytosis inhibitors. Positive control: no inhibitors. Clathrin-dependent pathway: Chlorpromazine (CPZ) 20 μM. Caveolin-dependent pathway: Filipin III 5 μg/mL. Macropinocytosis pathway: EIPA 20 μM. These concentrations were chosen following a dose-response assay for each inhibitor. Based upon the foregoing, NPs were taken up by the cells via clathrin-dependent and caveolin-dependent pathways. Membrane fusion is also involved, as indicated by 4° C. inhibition of DOX/DNA uptake.

FIG. 23 looks at EL4 DOX uptake after exposure to inhibitors NaN₃, PS2, Filipin III, EIPA, or 4° C. The inhibition studies suggest DOX is taken up by cells primarily via membrane fusion.

FIG. 24 provides confocal laser scanning microscope (CLSM) images of EL4 cells treated with DOX/DNA-Cy5 from 0 to 8 hours. The images indicate that DOX/DNA was taken up by EL4 cells over time. The images further suggest internalization of the nanoparticle, and not just DOX alone.

FIG. 25 presents a titration curve of DOX, DNA, and DOX/DNA using a weak base. Each solution's pH was brought down below 2 with 1 M HCl. The pH was measured after each addition of 100 μL or 20 μL 0.1 M NaOH. DNA pKa at 1 (phosphate), 6-7 (phosphate). DOX pKa at 7.34 (phenol), 8.46 (amine), 9.46 (estimated).

### DETAILED DESCRIPTION

As used herein and in the appended claims, the singular forms “a,” “an,” and “the” include plural referents unless the context clearly dictates otherwise. Thus, for example, reference to “a vector” includes a plurality of such vectors and reference to “the nucleic acid” includes reference to one or more nucleic acids and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstrom macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic my elogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In a particular embodiment, the cancer is selected from the group consisting of acute lymphoblastic leukemia, acute myeloblastic leukemia, bone sarcoma, breast cancer, endometrial cancer, gastric cancer, head and neck cancer, Hodgkin lymphoma, Non-Hodgkin lymphoma, liver cancer, kidney cancer, multiple myeloma, neuroblastoma, ovarian cancer, small cell lung cancer, soft tissue sarcoma, thyomas, thyroid cancer, transitional cell bladder cancer, uterine sarcoma, Wilms' tumor, and Waldenstrom macroglobulinemia.

The term “disorder” as used herein is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

The term “non-release controlling excipient” as used herein, refers to an excipient whose primary function do not include modifying the duration or place of release of the active substance from a dosage form as compared with a conventional immediate release dosage form.

The term “pharmaceutically acceptable carrier,” “pharmaceutically acceptable excipient,” “physiologically acceptable carrier,” or “physiologically acceptable excipient” as used herein, refers to a pharmaceutically-acceptable material, composition, or vehicle, such as a liquid or solid filler, diluent, excipient, solvent, or encapsulating material. Each component should be “pharmaceutically acceptable” in the sense of being compatible with the other ingredients of a pharmaceutical formulation. It should also be suitable for use in contact with the tissue or organ of humans and animals without excessive toxicity, irritation, allergic response, immunogenicity, or other problems or complications, commensurate with a reasonable benefit/risk ratio. Examples of “pharmaceutically acceptable carriers” and “pharmaceutically acceptable excipients” can be found in the following, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004.

The term “therapeutics that can associate or bind with DNA or RNA” as used herein refers to small molecules that can associate or bind with DNA or RNA and can be used to treat a disorder or disease in a subject, typically cancer. Examples of “therapeutics that can associate or bind with DNA or RNA” include but are not limited to, anthracyclines, such as aclarubicin, amrubicin, daunorubicin, doxorubicin, epirubicin, idarubicin, pirarubicin, valrubicin, and zorubicin; anthracenediones, such as mitoxantrone, and pixantrone; camptotheca compounds, such as belotecan, camptothecin, cositecan, exatecan, gimatecan, irinotecan, lurtotecan, rubitecan, silatecan, and topetecan; podophyllum compounds, like etoposide, and teniposide; bleomycin; actinomycin D; minor groove binders, such as duocarmycin A, adozelesin, bizelesin, and carzelesin; purine antagonists, such as cladribine, clofarabine, nelarbine, mercptopurine, tioguanine, and pentostatin; pyrimidine antagonists such as capecitabine, carmofur, doxifluridine, floxuridine, fluorouracil, tegafur, cytarabine, gemcitabine, azacytidine, and decitabine; folate antagonists, such as aminopterin, methotrexate, pemetrexed, and pralatrexate; alkylating agents, such as cyclophosphamide, ifosfamide, trofosfamide, chlorambucil, melphalan, prdednimustine, bendamustine, chlormethine, uramustine, carmustine, fotemustine, lomustine, nimustine, ranimustine, streptozocin, mannosulfan, treosulfan, carboquone, thiotepa, triaziquone, triethylenemelamine, carboplatin, cisplatin, dicycloplatin, nedaplatin, oxaliplatin, satraplatin, temozolomide, dacarbazine, mitobronitol, pipobroman, and procarbazine.

The term “release controlling excipient” as used herein, refers to an excipient whose primary function is to modify the duration or place of release of the active substance from a dosage form as compared with a conventional immediate release dosage form.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenicity, are commensurate with a reasonable benefit/risk ratio, and are effective for their intended use.

The terms “treat”, “treating” and “treatment”, as used herein, refers to ameliorating symptoms associated with a disease or disorder (e.g., cancer), including preventing or delaying the onset of the disease or disorder symptoms, and/or lessening the severity or frequency of symptoms of the disease or disorder.

The term “subject” as used herein, refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein. For example, a mammalian subject can refer to a human patient.

Therapeutics that can associate or bind with DNA or RNA have great potential for treating cancers and other diseases, but their inherent chemical structure can make them insoluble leading to poor bioavailability. Some of these therapeutics, while soluble, can lead to systemic toxicity, and can often be cleared too quickly from the body. Current solutions to these problems include delivering such compounds (e.g., anthracyclines) using a nanocarrier. Common disadvantages to these solutions, however, include very low drug loading, immunogenicity, poor therapeutic efficacy with slow clearance of the carriers, and substantial increase in cost. For example, DOXIL, a polyethylene glycolated (PEGylated) liposomal formulation of doxorubicin (DOX), experiences all of these limitations. Although it improves the safety profile of doxorubicin, it exhibits less efficacy than DOX. DOXIL's prolonged circulation in the bloodstream actually allows the immune system to develop antibodies against the PEGylated moieties on the particle. Thus, a major shortcoming of current solutions includes the biocompatibility of the nanocarrier. Moreover, current solutions do not have the ease of assembly. In direct contrast, the compositions and methods of the disclosure can be assembled in a straightforward manner, and are more cost effective. For example, HPMA-DOX (N-(2-hydroxypropyl) methyl acrylamide polymer-doxorubicin), another DOX nanocarrier, has a reported shorter circulation time (20.1 h) than compositions described herein, and has a more involved assembly process that would be difficult to scale to a commercial level. Others have synthesized nucleic acid systems to deliver chemotherapies (e.g., click nucleic acids for DOX and cytosine deaminase delivery), but such formulations are quite cost- and time-consuming and are unlikely to reach commercial stages. Furthermore, the PEGylation of such formulations will likely lead to similar immunogenicity issues that have previously reported for other PEGylated delivery systems, like DOXIL.

As shown in the in vitro and in vivo studies presented herein, use of nucleic acid fragments as a delivery vehicle for DOX resulted in improved safety and efficacy for the treatment of induced solid tumors in mice. In particular, the in vivo studies presented herein, DOX/DNA nanoparticle treatment improved survival and slowed tumor growth in comparison to DOX treatment alone. The foregoing favorable outcomes are likely the result prolonged circulation of DOX/DNA nanoparticles and controlled release of DOX from DNA, as evidenced by the 24 h, 48 h, and 72 h in vitro cytotoxicity studies and by the in vivo blood circulation study. Both means allow the DOX/DNA nanoparticles to exert a chemotherapeutic effect that is superior to DOX treatment alone, and superior to DOXIL treatment. DOXIL has been shown to be effective in reducing systemic toxicity effects, but does not result in an improved treatment outcome. Moreover, the PEGylation of DOXIL and the repeated administration of this chemotherapy formulation has been shown to result in immunogenicity. Other delivery vehicles in the field of nanotherapeutics can unfortunately be quite complex in their formulation and production, thus leading to difficulties in scalability. Both therapeutics and nucleic acids are already manufactured on the commercial scale. Thus, therapeutic/nucleic acid formulations, preparations and compositions are easy to produce and commercially scalable.

In a particular embodiment, the disclosure provides for a composition, preparation or formulation comprising one or more therapeutics that have been complexed with nucleic acids to form nanoparticles. Examples of therapeutics that can be complexed with nucleic acids to form nanoparticles include, but are not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, tricrynafen, chlorthalidone, furosenilde, musolimine, bumetanide, triamterene, amiloride, and spironolactone; anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; polyphenol compounds; polyketide compounds; prenyl-protein transferase inhibitors; and cyclosporins; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin. While the exemplary studies presented herein, clearly indicate that DOX/nucleic acid nanoparticles disclosed herein can be used to effectively treat cancer, it should be understood that any disease or disorder that is treatable by therapeutic agents is encompassed by this disclosure.

In a particular embodiment, the disclosure provides for a therapeutic composition, preparation or formulation comprising polyphenols that have been complexed with nucleic acids to form nanoparticles. Polyphenols are a structural class of mainly natural, but also synthetic or semisynthetic, organic chemicals characterized by the presence of large multiples of phenol structural units. The number and characteristics of these phenol structures underlie the unique physical, chemical, and biological (metabolic, toxic, therapeutic, etc.) properties of particular members of the class. Many polyphenols are micronutrients produced as secondary metabolites by dietary plants. Although these compounds display poor bioavailability (only a proportion of ingested amounts are absorbed and excretion is rapid), and complex pharmacodynamics and metabolism, they present therapeutic properties. A substantial body of evidence (epidemiological studies, animal studies and human clinical trials) indicate that polyphenols reduce a range of pathologies associated with cardiovascular disease including thrombosis (Navarro-Nunez et al., J Agric Food Chem. 2008; 56:2970-2976.), atherosclerosis (Chiva-Blanch et al., Am J Clin Nutr. 2012; 95:326-334.) and inflammation (Rieder et al., Br J Pharmacol. 2012; 167:1244-1258.), as well as displaying anti-cancer (Gali et al., Cancer Res. 1991; 51:2820-2825.) and neuroprotective (Gatson et al., J Trauma Acute Care Surg. 2013; 74:470-475) properties. The activities of these compounds are achieved via a range of mechanisms including their well-characterized antioxidant effects (Pignatelli et al., Atherosclerosis. 2006; 188:77-83), inhibition of intracellular kinase activity (Wright et al., Regen Med. 2012; 7:295-307), binding to cell surface receptors (Jacobson et al., Adv Exp Med Biol. 2002; 505:163-171) and disrupting the integrity of cell plasma membranes (Pawlikowska-Pawlega et al., Biochim Biophys Acta. 2007; 1768:2195-2204). Research on the application of polyphenols has increased especially in functional foods, nutraceutical, and pharmaceutical industries. However, one problem in human health is related to the effectiveness of polyphenols, which depends on preserving the stability, bioactivity, and bioavailability of the bioactive compounds. Additionally, the unpleasant taste of some phenolic compounds limits their use in pharmaceutical application. The encapsulation or complexation of polyphenols with nucleic acids disclosed herein, can effectively help to solve some of the drawbacks seen with the free polyphenol compounds. As the compositions and methods disclosed herein are directed to a platform-based polyphenol delivery system, it is expected that any type of polyphenol can be complexed or encapsulated by the nucleic acids disclosed herein. Exemplary examples of such polyphenol compounds, include but are not limited to, xanthohumols; flavanols, such as epicatechin, epigallocatechin, EGCG, and procyanidins; flavanones, such as hesperidin, and naringenin; flavones, such as apigenin, chrysin, and luteolin; flavonols, such as quercetin, kaempferol, myricetin, isorhamnetin, and galangin; isoflavonoids, such as genistein, and daidzein; phenolic acids, such as ellagic acid, gallic acid, ferulic acid, and chlorogenic acid; lignans, such as sesamin, and secoisolariciresinol diglucoside; stilbenes, such as resveratrol, pterostilbene, and piceatannol. Accordingly, the disclosure provides a platform technology that provides for formulations, compositions or preparations that allow for safe, efficient and controlled delivery of polyphenols in a subject to treat any number of diseases or disorders that are treatable by polyphenolic compounds. For example, numerous studies have demonstrated that polyphenols limit the incidence of coronary heart diseases (Renaud et al., Lancet. 1992; 339:1523-1526; Dubick et al., J Nutraceut Functional & Med Foods. 2001; 3:67-93; Nardini et al., Platelets. 2007; 18:224-243; and Vita et al., Am J Clin Nutr. 2005; 81:292-297); type II diabetes (Rizvi et al., Clin Exp Pharmacol Physiol. 2005; 32:70-75; Matsui et al., J Agric Food Chem. 2002; 50:7244-7248; Dembinska-Kiec et al., Br J Nutr 20. 2008; 99:109-117; and Chen et al., Eur J Pharmacol. 2007; 568:269-277); obstructive lung disease (Tabak et al., Am J Respir Crit Care Med. 2001; 164:61-64; and Woods et al., Am J Clin Nutr. 2003; 78:414-421); and neurodegenerative diseases (Ajami et al., Neuoroscience & Biobehavioral Reviews 2007; 73:39-47; and Mandel et al., Free Radical Biology and Medicine 2004; 37(3):304-317). It should be further noted that the preparations, compositions, or formulations disclosed herein are not just limited to the delivery of one particular polyphenol compound, as any number of polyphenol compounds can be complexed with nucleic acids disclosed herein to make polyphenol/nucleic acid nanoparticles.

Additionally, polyketide compounds can be complexed with the nucleic acids disclosed herein, or alternatively both polyketide and polyphenol compounds can be complexed with the nucleic acids disclosed herein. Polyketides are a large group of secondary metabolites which either contain alternating carbonyl and methylene groups (—CO—CH2-), or are derived from precursors which contain such alternating groups. Many polyketides have antimicrobial and immunosuppressive properties. Like with polyphenol compounds, polyketide compounds are capable of forming pi-pi stacking interactions with the nucleic acid species disclosed herein to form polyketide/nucleic acid nanoparticles.

In a particular embodiment, the disclosure provides for a composition, preparation or formulation comprising one or more therapeutics that can associate or bind with DNA or RNA disclosed are complexed with nucleic acids to form nanoparticles. Examples of therapeutics that can be complexed with nucleic acids to form nanoparticles include, but are not limited to, anthracyclines, such as aclarubicin, amrubicin, daunorubicin, doxorubicin, epirubicin, idarubicin, pirarubicin, valrubicin, and zorubicin; anthracenediones, such as mitoxantrone, and pixantrone; camptotheca compounds, such as belotecan, camptothecin, cositecan, exatecan, gimatecan, irinotecan, lurtotecan, rubitecan, silatecan, and topetecan; podophyllum compounds, like etoposide, and teniposide; bleomycin; actinomycin D; minor groove binders, such as duocarmycin A, adozelesin, bizelesin, and carzelesin; purine antagonists, such as cladribine, clofarabine, nelarbine, mercptopurine, tioguanine, and pentostatin; pyrimidine antagonists such as capecitabine, carmofur, doxifluridine, floxuridine, fluorouracil, tegafur, cytarabine, gemcitabine, azacytidine, and decitabine; folate antagonists, such as aminopterin, methotrexate, pemetrexed, and pralatrexate; alkylating agents, such as cyclophosphamide, ifosfamide, trofosfamide, chlorambucil, melphalan, prdednimustine, bendamustine, chlormethine, uramustine, carmustine, fotemustine, lomustine, nimustine, ranimustine, streptozocin, mannosulfan, treosulfan, carboquone, thiotepa, triaziquone, triethylenemelamine, carboplatin, cisplatin, dicycloplatin, nedaplatin, oxaliplatin, satraplatin, temozolomide, dacarbazine, mitobronitol, pipobroman, and procarbazine. In a certain embodiment, the one or more therapeutics that can associate or bind with DNA or RNA is selected from anthracyclines, such as aclarubicin, amrubicin, daunorubicin, doxorubicin, epirubicin, idarubicin, pirarubicin, valrubicin, and zorubicin; anthracenediones, such as mitoxantrone, and pixantrone; camptotheca compounds, such as belotecan, camptothecin, cositecan, exatecan, gimatecan, irinotecan, lurtotecan, rubitecan, silatecan, and topetecan; podophyllum compounds, like etoposide, and teniposide; bleomycin; actinomycin D; minor groove binders, such as duocarmycin A, adozelesin, bizelesin, and carzelesin. In a further embodiment, the one or more therapeutics that can associate or bind with DNA or RNA comprises aclarubicin, amrubicin, daunorubicin, doxorubicin, epirubicin, idarubicin, pirarubicin, valrubicin, and/or zorubicin. In another embodiment, the one or more therapeutics that can associate or bind with DNA or RNA comprises mitoxantrone, topetecan, etoposide, teniposide, bleomycin, actinomycin D, and/or duocarmycin A.

As the compositions and methods disclosed herein are directed to a platform-based therapeutic delivery system, it is expected that any type of therapeutic compound that associates or binds with DNA or RNA can be complexed or encapsulated by the nucleic acids disclosed herein. Exemplary examples of such therapeutic compounds, include but are not limited to, anthracyclines, such as aclarubicin, amrubicin, daunorubicin, doxorubicin, epirubicin, idarubicin, pirarubicin, valrubicin, and zorubicin; anthracenediones, such as mitoxantrone, and pixantrone; camptotheca compounds, such belotecan, camptothecin, cositecan, exatecan, gimatecan, irinotecan, lurtotecan, rubitecan, silatecan, and topetecan; podophyllum compounds, like etoposide, and teniposide; bleomycin; and actinomycin D. Accordingly, the disclosure provides for a platform technology that can be used for formulations, compositions or preparations for safe, efficient and controlled delivery of therapeutics that can associate or bind with DNA or RNA in a subject to treat any number of diseases or disorders that are treatable by such therapeutics. While the exemplary studies presented herein, clearly indicate that DOX/nucleic acid nanoparticles disclosed herein can be used to effectively treat cancer, it should be understood that any disease or disorder that is treatable by therapeutics that can associate or bind with DNA or RNA is encompassed by this disclosure. It should be further noted that the preparations, compositions, or formulations disclosed herein are not just limited to the delivery of one particular therapeutic that associates or binds with DNA or RNA, as any number of therapeutics that can associate or bind with DNA or RNA can be complexed with nucleic acids disclosed herein to make therapeutic/nucleic acid nanoparticles.

In regards to the nucleic acid component of the therapeutic/nucleic acid nanoparticles, any type and length of nucleic acid species may be used to complex with the therapeutics that can associate or bind with DNA or RNA. Namely, the nucleic acid species should be capable of forming pi-pi stacking interactions with therapeutics that can associate or bind with DNA or RNA. While DNA was used in the studies presented herein, it is envisaged DNA, RNA, DNA-RNA hybrids, or mixtures thereof could be used to form therapeutic/nucleic acid nanoparticles disclosed herein. Moreover, for purposes of this disclosure “nucleic acids” include nucleic acid analogues. Nucleic acids are chains of nucleotides, which are composed of three parts: a phosphate backbone, a pentose sugar, either ribose or deoxyribose, and one of four nucleobases. A nucleic acid analogue may have any of these altered.

DNA, abbreviation of deoxyribonucleic acid, is an organic chemical of complex molecular structure that is found in all prokaryotic and eukaryotic cells and in many viruses. DNA codes genetic information for the transmission of inherited traits. Each strand of a DNA molecule is composed of a long chain of monomer nucleotides. The nucleotides of DNA consist of a deoxyribose sugar molecule to which is attached a phosphate group and one of four nitrogenous bases: two purines (adenine and guanine) and two pyrimidines (cytosine and thymine). The nucleotides are joined together by covalent bonds between the phosphate of one nucleotide and the sugar of the next, forming a phosphate-sugar backbone from which the nitrogenous bases protrude. One strand is held to another by hydrogen bonds between the bases; the sequencing of this bonding is specific—i.e., adenine bonds only with thymine, and cytosine only with guanine. The configuration of the DNA molecule is highly stable, allowing it to act as a template for the replication of new DNA molecules, as well as for the production (transcription) of the related RNA (ribonucleic acid) molecule.

RNA, abbreviation of ribonucleic acid, is a complex compound of high molecular weight that functions in cellular protein synthesis and replaces DNA (deoxyribonucleic acid) as a carrier of genetic codes in some viruses. RNA consists of ribose nucleotides (nitrogenous bases appended to a ribose sugar) attached by phosphodiester bonds, forming strands of varying lengths. The nitrogenous bases in RNA are adenine, guanine, cytosine, and uracil, which replaces thymine in DNA. The ribose sugar of RNA is a cyclical structure consisting of five carbons and one oxygen. The presence of a chemically reactive hydroxyl (—OH) group attached to the second carbon group in the ribose sugar molecule makes RNA prone to hydrolysis. This chemical lability of RNA, compared with DNA, which does not have a reactive —OH group in the same position on the sugar moiety (deoxyribose), is thought to be one reason why DNA evolved to be the preferred carrier of genetic information in most organisms. In a particular embodiment, this reactive —OH group of RNA may be replaced by a less reactive —O-alkyl group or halide group, to make the RNA resistant to the action of RNAses.

DNA-RNA hybrids are abundant in human cells. They form during transcription when nascent RNA is in close proximity to its DNA template. The resulting RNA/DNA hybrids and the displaced single-stranded (ss) DNA are called R-loops. RNA/DNA hybrids are structurally different and more stable than the corresponding double-stranded DNAs. RNA/DNA hybrids are found in origins of replication, immunoglobulin class-switch regions, and transcription complexes. RNA/DNA hybrids do not adopt the traditional B-conformation of DNA or A-conformation of RNA but occur as mixtures or heterogenous duplexes.

For purposes of this disclosure, fragments of nucleic acids can result from the enzymatic cleavage or physical breakage of naturally occurring nucleic acids; chemical synthesis of various sizes of nucleic acids; or some combination thereof Δny naturally occurring nucleic acid may be used, including nucleic acids from any species, from prokaryotes, from eukaryotes, from fungi, etc. In a particular embodiment, the nucleic acid fragments are from salmon DNA. Further, the sizes/lengths of nucleic acid fragments can be varied to suit particular therapeutic being used. For example, fragments of nucleic acids can have a length of 20 nt, 30 nt, 40 nt, 50 nt, 60 nt, 70 nt, 80 nt, 90 nt, 100 nt, 110 nt, 120 nt, 130 nt, 140 nt, 150 nt, 160 nt, 170 nt, 180 nt, 190 nt, 200 nt, 250 nt, 300 nt, 350 nt, 400 nt, 450 nt, 500 nt, 550 nt, 600 nt, 650 nt, 700 nt, 750 nt, 800 nt, 850 nt, 900 nt, 950 nt, 1,000 nt, 1,500 nt, 2,000 nt, 2,500 nt, 3,000 nt, 3,500 nt, 4,000 nt, 4,500 nt, 5,000 nt, 5,500 nt, 6,000 nt, 6,500 nt, 7,000 nt, 7,500 nt, 8,000 nt, 8,500 nt, 9,000 nt, 9,500 nt, 10,000 nt, or a range of lengths that is between or includes any two of the foregoing lengths (e.g., 20 nt to 10,000 nt, 50 nt to 2,000 nt, etc.). The sequence of the nucleic acid may be random or be selected to have a desired sequence. In the later case, sequences may be selected to target transcription factors (TFs), TLRs, or other DNA or RNA-binding proteins; or are aptamers. In such a case, the therapeutic/nucleic acid nanoparticles may be targeted to certain tissue, organs, or tumors, via selection of a particular sequence or a ligand to tumor-specific antigens. Ligands to tumor-specific antigens are commercially available from a variety of vendors, and therefore do not have to be generated de novo (e.g., see Elabscience, Santa Cruz biotechnology, Biospacific, Novus Biologicals, etc.). In a particular embodiment, the ligand attached to the therapeutic agent/nucleic acid nanoparticles binds to a tumor specific antigen selected from alphafetoprotein (AFP), carcinoembryonic antigen (CEA), CA-125, CA15-3, CA19-9, MUC-1, epithelial tumor antigen (ETA), tyrosinase, melanoma-associated antigen (MAGE), abnormal products of ras or p53, CTAG1B, MAGEA1, and HER2/neu. The ligand that binds to the tumor-specific antigen should have bind to the target antigen with high affinity (K_{d}<10 nM) for efficient uptake into target tumor cells and it should be minimally immunogenic. In a further embodiment, the ligand that binds to the tumor-specific antigen is attached to a therapeutic agent/nucleic acid nanoparticle disclosed herein via a use of a cleavable linker (acid-labile linkers, protease cleavable linkers, and disulfide linkers). Acid-labile linkers are designed to be stable at pH levels encountered in the blood, but become unstable and degrade when the low pH environment in lysosomes is encountered. Protease-cleavable linkers are also designed to be stable in blood/plasma, but rapidly release free drug inside lysosomes in cancer cells upon cleavage by lysosomal enzymes. They take advantage of the high levels of protease activity inside lysosomes and include a peptide sequence that is recognized and cleaved by these proteases, as occurs with a dipeptide Val-Cit linkage that is rapidly hydrolyzed by cathepsins. A third type of linker that can be used to attach the ligand to the therapeutic agent/nucleic acid nanoparticle contains a disulfide linkage. This linker exploits the high level of intracellular reduced glutathione to release free drug inside the cell. Reagents, like Traut's reagent (2-iminothiolane), MBS (3-maleimidobenzoic acid N-hydroxysuccinimide ester), and SATA (N-succinimidyl S-acetylthioacetate) can convert such primary amine groups to sulfhydryls, which can then form disulfide bonds with ligands comprising cysteine residues. Other reagents, like SPDP (N-succinimidyl 3-(2-pyridyldithio) propionate), SMCC (succinimidyl 4-(N-maleimidomethyl) cyclohexane-1-carboxylate), and Sulfo-SMCC can be used as linkers for attaching ligands to nucleic acids of the therapeutic agent/nucleic acid nanoparticles. Examples of how to use of such groups for attaching ligands to the therapeutic agent/nucleic acid nanoparticles can be found on the worldwide web at labome.com/method/Antibody-Conjugation.html, and the references cited therein, including Safdari et al., Monoclon Antib Immunodiagn Immunother. 2013 32:409-12; Joosten V et al., Microb Cell Fact 2003 2:1; Winter et al., Trends Pharmacol Sci. 1993 14:139-43; Arbabi et al., Front Immunol. 2017 8:1589; Brinkley et al., Bioconjug Chem. 1992 3:2-13; Vlasak et al MAbs. 2011 3:253-63; Ducancel et al MAbs. 2012 4:445-57; McCombs et al., AAPS J. 2015; 17:339-51; Hondal R., Protein Pept Lett. 2005 12:757-64; Zimmerman et al., Bioconjug Chem. 2014 25:351-61; Traut et al., Biochemistry. 1973 12:3266-73; Knight P., Biochem 1 1979 179:191-7; Carlsson et al., Biochem 1 1978 173:723-37; Peeters J et al., Immunol Methods. 1989 120:133-43; Hashida et al., J Appl Biochem. 1984 6:56-63; Avrameas et al., Immunochemistry. 1971 8:1175-9; Richards et al., J Mol Biol. 1968 37:231-3; Chandler et al., J Immunol Methods. 1982 53:187-94; Coulepis et al., J Clin Microbiol. 1985 22:119-24; White et al., J Clin Microbiol. 1989 27:2300-4; Liu et al., J Immunol Methods. 2000 234:P153-67; Tian et al., Bioconjug Chem. 2015 26:1144-55; Vira et al., Anal Biochem. 2010 402:146-50; Szabo et al., Biophys 1 2018 114:688-700; Hagan et al., Lanthanide-Anal Bioanal Chem. 2011 400:2847-64; Han et al. Nat Protoc. 2018 13:2121-2148; Bottrill et al., Chem Soc Rev. 2006 35:557-71; Ye et al., J Clin Lab Anal. 2014 28:335-40; Fernandez Moreira et al., Analyst. 2010 135:42-52; Brouwers et al., J Nucl Med. 2004 45:327-37; Vera et al., Nucl Med Biol. 2012 39:3-13; Stein et al., J Nucl Med. 2001 42:967-74; Bratthauer G., Methods Mol Biol. 2010 588:257-70; Engle et al., Science. 2019 364:1156-1162; Sano et al., Science. 1992 258:120-2; Malou et al., Trends Microbiol. 2011 19:295-302; Cardoso et al., Curr Med Chem. 2012; 19:3103-27; East et al., Methods Mol Biol. 2014 1199:67-83; Tan et al., Nanomaterials (Basel). 2015 5:1297-1316; Geng et al., Bioconjug Chem. 2016 27:2287-2300; Pecanha et al., J Immunol. 1991 146:833-9; Pecanha et al., Immunol. 1993 150:2160-8; and Chen Y., Methods Mol Biol. 2013 1045:267-73, the disclosures of which are incorporated herein.

While the exemplary DOX/DNA nanoparticles disclosed in the studies presented herein polydisperse due to the nature of nucleic acid used; it is expected that monodisperse nanoparticles could be made based upon the selection of the nucleic acid. Such monodisperse nanoparticles may impart a more improved therapeutic outcome. Moreover, the nucleic acid making up the nanoparticles disclosed herein could be complexed using a cationic molecule (e.g., PTD domains) to provide or improve the controlled release properties of the nanoparticles (by minimizing degradation due to nucleases). Additionally, the hygroscopic nature of nucleic acids can be utilized to make therapeutic-loaded hydrogels; and the nucleic acids can be conjugated with proteins (e.g., thymosin-α 1) to provide for multi-modal approaches in treating a disease or disorder with the nanoparticles disclosed herein. For example, the therapeutic/nucleic acid nanoparticles can be conjugated with an immune enhancing protein such as thymosin-α 1 for a multi-modal approach by priming the immune system to fight against cancer while at the same time delivering an anticancer therapeutic compound.

The therapeutics may be complexed with nucleic acids at a certain weight to weight (wt/wt) ratio to form nanoparticles. For example, the nucleic acid fragments are complexed with the one or more therapeutic compounds at a wt/wt ratio of about 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, or a range that includes or is between any two of the foregoing ratios, including fractional increments thereof (e.g., 2:1 to 10:1, 4:1 to 7:1, 4.5:1 to 6.5:1, etc.). In a particular embodiment, the nucleic acid fragments are complexed with the one or more therapeutic compounds at a wt/wt ratio of about 6:1. The size of the therapeutic/nucleic acid nanoparticles can also be controlled based upon the concentration of the starting materials, reaction parameters (e.g., temperature, time, etc.), and addition of agents (e.g., surfactants, salts, etc.). In a particular embodiment, the size of the therapeutic/nucleic acid nanoparticles are about 10 nm, 12 nm, 14 nm, 15 nm, 16 nm, 18 nm, 20 nm, 22 nm, 24 nm, 25 nm, 26 nm, 28 nm, 30 nm, 32 nm, 34 nm, 35 nm, 36 nm, 38 nm, 40 nm, 42 nm, 44 nm, 45 nm, 46 nm, 48 nm, 50 nm, 52 nm, 54 nm, 55 nm, 56 nm, 58 nm, 60 nm, 62 nm, 64 nm, 65 nm, 66 nm, 68 nm, 70 nm, 72 nm, 74 nm, 75 nm, 76 nm, 78 nm, 80 nm, 82 nm, 84 nm, 85 nm, 86 nm, 88 nm, 90 nm, 92 nm, 94 nm, 95 nm, 96 nm, 98 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, or a range that includes or is between any two of the foregoing ratios, including fractional increments thereof (e.g., 20 nm to 200 nm, 50 nm to 100 nm, etc.). In a particular embodiment, the size of the therapeutic/nucleic acid nanoparticles are about 70 nm. The nanoparticles can have any shape, including generally spherical, ovoid, cubic, hexagonal, prism, rod, helical, triangular, star, or irregularly shaped.

In a certain embodiment, the disclosure provides for a pharmaceutical composition which comprises a therapeutic/nucleic acid nanoparticle disclosed herein. The pharmaceutical composition can be formulated into a form suitable for administration to a subject including the use of carriers, excipients, additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, cryoprotectants, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered at a therapeutically effective amount either locally or systemically. As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically, the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieved.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

In a particular embodiment, the therapeutic/nucleic acid nanoparticles disclosed herein can be administered in combination with anti-cancer agents known in the art to treat a subject with cancer. The therapeutic/nucleic acid nanoparticles disclosed herein can be administered, concurrently or sequentially, with anti-cancer agents to treat a subject with cancer. Use of the therapeutic/nucleic acid nanoparticles of the disclosure with the anti-cancer agents provides a multimodal therapy that can provide a more effective treatment of a cancer than use of the anticancer agent alone or use of the therapeutic/nucleic acid nanoparticles alone. Examples, of anticancer agents that can be used with the therapeutic/nucleic acid nanoparticles disclosed herein include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included anticancer agents are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above. In a particular embodiment, the therapeutic/nucleic acid nanoparticles disclosed herein are used in combination of one or more anticancer agents selected from cyclophosphamide, tamoxifen, tegafur, paclitaxel, apatinib, cisplatin, docetaxel, 5-fluorouracil, capecitabine, carboplatin, vinorelbine, capecitabine, gemcitabine, ixabepilone, eribulin, ifosfamide, rituximab, vincristine, prednisone, bleomycin, and dacarbazine.

For use in the therapeutic or biological applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more therapeutic/nucleic acid nanoparticles described herein, optionally in a composition or in combination with another agent (e.g., mRNA and/or ssRNA) as disclosed herein. The container(s) optionally have a sterile access port (for example the container can be an intravenous solution bag or a vial having a stopper pierceable by a hypodermic injection needle). Such kits optionally comprise an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself, a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The disclosure further provides that the methods and compositions described herein can be further defined by the following aspects (aspects 1 to 29):

1. A composition comprising one or more therapeutic compounds that are complexed with nucleic acid fragments to form nanoparticles, wherein the one or more therapeutic compounds are small molecules that can associate or bind with DNA or RNA.

2. The composition of aspect 1, wherein the nucleic acid fragments are complexed with the one or more therapeutic compounds at a wt/wt ratio of 2:1 to 10:1.

3. The composition of aspect 1 or aspect 2, wherein the nucleic acid fragments are complexed with the one or more therapeutic compounds at a wt/wt ratio of 4:1 to 7:1.

4. The composition of any one of the preceding aspects, wherein the nucleic acid fragments are complexed with the one or more therapeutic compounds at a wt/wt ratio of about 6:1.

5. The composition of any one of the preceding aspects, wherein the nanoparticles are from 20 nm to 200 nm in size.

6. The composition of any one of the preceding aspects, wherein the nanoparticles are from 50 nm to 100 nm in size.

7. The composition of any one of the preceding aspects, wherein the one or more therapeutic compounds comprises anthracyclines, anthracenediones, camptotheca compounds, podophyllum compounds, minor groove binders, bleomycin, and/or actinomycin D.

8. The composition of any one of the preceding aspects, wherein the one or more therapeutic compounds comprises aclarubicin, doxorubicin, daunorubicin, idarubicin, epirubicin, amrubicin, pirarubicin, valrubicin, and/or zorubicin.

9. The composition of any one of the preceding aspects, wherein the one or more therapeutic compounds comprises doxorubicin.

10. The composition of any one of the preceding aspects, wherein the one or more therapeutic compounds comprises mitoxantrone, topetecan, etoposide, teniposide, bleomycin, actinomycin D, and/or duocarmycin A.

11. The composition of any one of the preceding aspects, wherein one or more of the nucleic acid fragments comprise a ligand that targets the nanoparticles to specific cells, tissue, organs, or tumors.

12. The composition of any one of the preceding aspects, wherein the nucleic acid fragments comprise fragments of naturally occurring DNA, RNA and/or DNA-RNA hybrids.

13. The composition of any one of the preceding aspects, wherein the nucleic acid fragments comprise chemically synthesized DNA, RNA and/or DNA-RNA hybrids of differing nucleotide lengths.

14. The composition of any one of the preceding aspects, wherein the RNA has been modified to replace the 2′ ribose hydroxyl group with an —O-alkyl group or a halide.

15. The composition of any one of the preceding aspects, wherein the nucleic acid fragments are DNA fragments.

16. The composition of any one of the preceding aspects, wherein the DNA fragments are from salmon DNA.

17. The composition of any one of the preceding aspects, wherein the nucleic acid fragments are from 20 nt to 10,000 nt in length.

18. The composition of any one of the preceding aspects, wherein the nucleic acid fragments are from 50 nt to 2,000 nt in length.

19. The composition of any one of the preceding aspects, wherein the composition comprises nanoparticles of one or more therapeutic compounds complexed with DNA fragments from 50 nt to 2,000 nt in length.

20. The composition of any one of the preceding aspects, wherein the one or more therapeutic compounds is selected from aclarubicin, doxorubicin, daunorubicin, idarubicin, epirubicin, amrubicin, pirarubicin, valrubicin, and/or zorubicin.

21. The composition of any one of the preceding aspects, wherein the one or more therapeutic compounds is doxorubicin.

22. A pharmaceutical composition comprising the composition of any one of the preceding aspects, and a pharmaceutically acceptable carrier, diluent, and/or excipient.

23. The pharmaceutical composition of aspect 22, wherein the pharmaceutical composition is formulated for parenteral delivery.

24. A method of treating a subject having a cancer in need of treatment thereof, comprising: administering to the subject an effective amount of the pharmaceutical composition of aspect 22 or aspect 23.

25. The method of aspect 24, wherein the cancer is selected from acute lymphoblastic leukemia, acute myeloblastic leukemia, bone sarcoma, breast cancer, endometrial cancer, gastric cancer, head and neck cancer, Hodgkin lymphoma, Non-Hodgkin lymphoma, liver cancer, kidney cancer, multiple myeloma, neuroblastoma, ovarian cancer, small cell lung cancer, soft tissue sarcoma, thyomas, thyroid cancer, transitional cell bladder cancer, uterine sarcoma, Wilms' tumor, and Waldenstrom macroglobulinemia.

26. A method of treating a human subject having a cancer in need of treatment thereof, comprising: administering an effective amount of the composition of any one of aspects 1 to 21 to the subject.

27. The method of aspect 26, wherein the cancer is selected from acute lymphoblastic leukemia, acute myeloblastic leukemia, bone sarcoma, breast cancer, endometrial cancer, gastric cancer, head and neck cancer, Hodgkin lymphoma, Non-Hodgkin lymphoma, liver cancer, kidney cancer, multiple myeloma, neuroblastoma, ovarian cancer, small cell lung cancer, soft tissue sarcoma, thyomas, thyroid cancer, transitional cell bladder cancer, uterine sarcoma, Wilms' tumor, and Waldenstrom macroglobulinemia.

28. The method of aspect 26 or aspect 27, wherein the method further comprises administering to the subject with one or more anticancer agents selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and/or HDAC inhibitors.

29. The method of any one of the aspects 26 to 28, wherein the method further comprises administering to the subject with one or more anticancer agents selected from mitoxantrone, topetecan, etoposide, teniposide, bleomycin, actinomycin D, and duocarmycin A.

The following examples are intended to illustrate but not limit the disclosure. While they are typical of those that might be used, other procedures known to those skilled in the art may alternatively be used.

### Examples

Materials. Doxorubicin (DOX), and ethidium bromide were purchased from Thermo Fisher Scientific (Waltham, Mass.). Deoxyribonucleic acid (DNA) (50-2000 nucleotide fragments with a MW range of 16.88 kDa — 1350 kDa) was provided by Pharma Research Products Co., Ltd (Seongnam, Korea). 3-(4,5-dimethyl-2-thiazolyl)-2,5-diphenyltetrazolium bromide (MTT) was purchased from Millipore Sigma (Burlington, Mass.). ULYSIS™ Alexa Fluor™ 488 Nucleic Acid Labeling Kit was purchased from Thermo Fisher Scientific. Label IT® Nucleic Acid Labeling Kit, Cy®5 was purchased from Mirus Bio.EL4 cells (ATCC, Rockville, Md.) were cultured in Dulbecco's modification of Eagle's medium (DMEM) (MediaTech, Manassas, Va.) with 10% fetal bovine serum (FBS) (Atlanta Biologicals, Flowery Branch, Ga.) and 1% antibiotics (100 units/mL penicillin; 100 μg/mL streptomycin) (Gibco, Grand Island, N.Y.). All materials were used as purchased.

DOX Quenching Using DNA. To optimize the encapsulation efficiency, a quenching study was performed. Fluorescence spectra of DNA:DOX ratios between 1-100 were first measured, then fluorescence spectra of DNA:DOX ratios between 1-10 were measured. Excitation was performed at 490 nm, while emission was performed at 590 nm. Fluorescence was read using a Xenon laser and a Synergy H1 Hybrid Multi-Mode Reader (Biotek Instruments, Winooski, Vt.).

Preparation of DOX/DNA nanoparticles. Briefly, DNA was added to DOX at a 6:1 w/w ratio. Time was allotted for self-assembly. Finally, phosphate buffered saline (PBS) was added to the mixture, and a further 30 minutes was allotted for ionic stabilization of the complex.

An equal volume of DNA was pipetted into an equal volume of DOX and was pipetted up and down to mix. The reactants and reaction were kept at 70° C. Then, it was left to sit for 15 minutes to allow for self-assembly. Then, an equal volume of 2×PBS was pipetted into the reaction, and was pipetted up and down to mix. After allowing for a 30-minute rest period, the DOX/DNA nanoparticles were used for further experimentation. For small volumes under 25 mL, a 96-well plate was used. For large volumes over 25 mL, round bottom flasks were used, and mixing was induced by magnetic stir bars which are spun at 500 RPM on a multi-plate stirrer (IKA Works, Inc., Wilmington, N.C.). In this case, DNA was added drop-wise to DOX by a glass pipette. To verify formation of the nanoparticles, transmission electron microscope (TEM) was used to take images of DOX/DNA.

Transmission Electron Imaging of DOX/DNA and DNA. An equal volume of DNA was pipetted into an equal volume of DOX and was pipetted up and down to mix. The reactants and reaction are kept at 70° C. Then, it was left to sit for 15 minutes to allow for self-assembly. Then, an equal volume of 2×PBS was pipetted into the reaction, and was pipetted up and down to mix. A 30-minute rest period was provided before using the DOX/DNA nanoparticle for further experimentation. For small volumes under 25 mL, a 96-well plate was used. For large volumes over 25 mL, round bottom flasks were used, and mixing was induced by magnetic stir bars which are spun at 500 RPM on a multi-plate stirrer (IKA Works, Inc., Wilmington, N.C.). In this case, DNA was added drop-wise to DOX by a glass pipette. To verify that nanoparticles were made, a transmission electron microscope (TEM) was used to take images of DOX/DNA.

DOX/DNA solutions (10 μL) were dropped on a carbon-coated grid (Thermo Fisher Scientific) and dried overnight at room temperature. The morphology and size of the nanoparticles were observed under a JEOL 2800 transmission electron microscope (JEOL, Peabody, Mass.) at 200 kV.

DNA Degradation in 10% FBS/PBS. A 1% (w/v) agarose gel in 1×Tris Acetate EDTA (TAE) containing 1 μg/mL ethidium bromide was used to characterize the degradation of DNA in 10% FBS/PBS over 48 hours. DNA was incubated with serum-containing PBS at 37° C. over time. Samples were stored in −20° C. to stop enzymatic degradation from nucleases at each time point. The concentration of DNA=100 μg/mL. The gel was run at 100 V for 40 minutes and DNA was imaged on a UV transilluminator (Fotodyne, Heartland, Wis.).

Binding Kinetics. Binding kinetics of DOX with DNA was measured by observing fluorescence of DOX/DNA based on the concentration of DOX. Fluorescence of DOX/DNA was measured as DOX was increased. DNA remained constant at 400 μg/mL. Fluorescence was measured in using the Multi-Mode reader. Binding kinetics of DOX with DOX/DNA was studied in PBS, serum-containing PBS, or FBS.

EL4 Cytotoxicity. EL4 cells (ATCC, Rockville, Md.) were plated at 10 k cells per well in a 96-well plate. The plated cells were treated in triplicate for 24, 48, or 72 hours with a range of concentrations of 0.001 μg/mL DOX or DOX equivalent to 10 μg/mL, followed by cellular viability analysis via MTT assay. Cells were incubated at 37° C., 5% CO₂, and 100% humidity. Absorbance was read at 571 nm.

Endocytosis Inhibition. EL4 DOX uptake from DOX or DOX/DNA treatment after exposure to inhibitors NaN₃ (120 mM), PS2 (12 μg/mL), Filipin III (5 μg/mL), CPZ (20 μM), EIPA (20 μg/mL), or 4° C. was measured using flow cytometry or spectrofluorimetry. Briefly, cells were primed for 15 minutes with the inhibitors before being treated for 1 hour with DOX or DOX/DNA. Cells containing DOX or DOX/DNA were counted with the flow cytometer using yellow fluorescence, and cells containing DOX/DNA were counted using spectrofluorimetry. Fluorescence was measured using a Guava® easyCyte™ Flow Cytometer (Millipore Sigma, Burlington, Mass.) or the Multi-Mode reader (Biotek Instruments, Winooski, Vt.).

Confocal Imaging. EL4 cells were first plated on a 35 mm Ibidi μ-Dish (Ibidi USA Inc., Fitchburg, Wis.) at 200k cells per mL. Then, the nuclei were stained and allowed to incubate for 15 minutes. The cells were spun and washed with DPBS before treating with DOX/DNA-Cy5 for three hours. The cells were finally spun at 500×g and washed with DPBS before placing in DMEM and imaged live using the Leica TCS SP8 Confocal Laser-Scanning Microscope (Leica Microsystems, Buffalo Grove, Ill.).

In vivo Studies. All animal studies were conducted using IACUC-approved procedures. EL4 tumors were established in the right rear flank of 6-12 week old female C57BL/6/027 mice (Charles River Laboratories, Wilmington, Mass., USA) by subcutaneously injecting 1E6 EL4 cells in approximately 100 μL of PBS. Once tumors were visible and measurable at 2 mm, the mice were administered treatment via tail vein injection. Tumor size was measured using a digital caliper, and the tumor volume was calculated using the following equation: V=W²L/2, where V is tumor volume, W is the width of the tumor, and L is the length of the tumor. Mice were given food and water ad libitum.

Pharmacokinetics. A pharmacokinetics study was performed on EL4-challenged C57BL/6 mice treated i.v. with either 20 mg/kg DOX or 20 mg/kg DOX equiv. of DOX/DNA in 6-8-week-old female mice, n=3. Blood samples were collected from the saphenous vein of the mice at each time point, spun down in a serum collection tube, and the resultant supernatant was analyzed in acidified alcohol for DOX fluorescence using the Multi-Mode reader.

Hematotoxicity and Liver Enzyme Panel. The mice used in this study were 6-12 week-old female C57BL/6/027 mice (Charles River Laboratories, Wilmington, Mass., USA). The mice were administered treatment via tail vein injection. After 24 h, complete blood count (CBC), and liver enzyme levels were measured. Blood for CBC was collected by saphenous vein collection, mixed with EDTA, and analyzed with a hematology analyzer for white blood cells (WBC), red blood cells (RBC), hemoglobin (Hgb), platelets (Plt) and hematocrit (HCT). For the liver enzyme panel, serum was isolated from blood and sent to IDEXX Laboratories, Inc. (Westbrook, Me.) for alkaline phosphatase (ALP), aspartate aminotransferase (AST), alanine aminotransferase (ALT), and total bilirubin analysis.

Binding Kinetics. Binding kinetics of DOX with DNA was measured by observing fluorescence of DOX/DNA based on the concentration of DOX. Fluorescence of DOX/DNA was measured as [DOX] was increased. [DNA] remained constant at 400 μg/mL. Fluorescence was measured in using the Multi-Mode reader. Binding kinetics of DOX with DOX/DNA was studied in PBS, serum-containing PBS, or FBS.

Biodistribution. DOX accumulation in organs and the tumor were measured after a 20 mg/kg i.v. administration of DOX or DOX/DNA (20 mg/kg DOX equivalent) in EL4-challenged C57BL/6 mice (Female, 6-8 weeks old) at 1, 3, 6, and 12 hours, n=5. Briefly, organs and tumor were harvested, cryopulverized, and homogenized in acidified alcohol before centrifugation. The resulting supernatant was analyzed for DOX fluorescence using the Multi-Mode reader.

Acute Toxicity. Acute toxicity in C57BL/6 mice (Female, 6-8 weeks) was observed at 24 h post injection with doses administered from 10 to 40 mg/kg of DOX or DOX equivalent, n=7.

Tumor Growth and Survival. Tumor growth and survival of EL4-challenged mice were tracked regularly for 30 days after i.v. treatment of DOX, DOX/DNA, or DOXIL with a range of doses (6-12-week-old female mice), n=5. Initial tumor challenge consisted of 1E6 EL4 cells injected subcutaneously in the right rear flank of the mice. When tumor growth of 2 mm was measurable, the treatment was administered in the tail vein. Mice were euthanized when tumors exceeded 15 mm, when tumor lesions appeared, or when weight fell below 75% initial weight.

Repeated Administration Tumor Growth and Survival. Tumor growth, weight, and survival of EL4-challenged mice (6 weeks, female) were tracked over 22 days after the initial i.v. treatment of DOX. DOX/DNA, or DOXIL at 20 mg/kg on day 0. Subsequently, on days 7 and 14, 20 mg/kg treatments of DOX, DOX/DNA, or DOXIL were administered, n=5. Initial tumor challenge consisted of 1E6 EL4 cells injected subcutaneously in the right rear flank of the mice. When tumor growth of 2 mm was measurable, the treatment was administered in the tail vein. Mice were euthanized when tumors exceeded 15 mm, when tumor lesions appeared, or when weight fell below 75% initial weight.

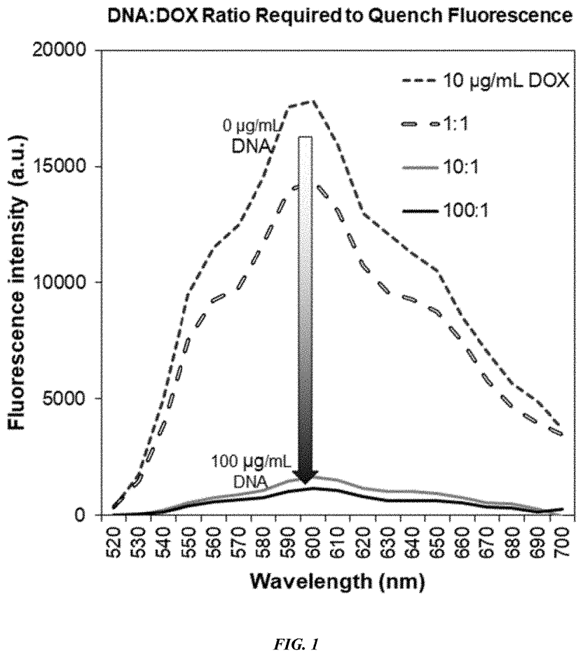

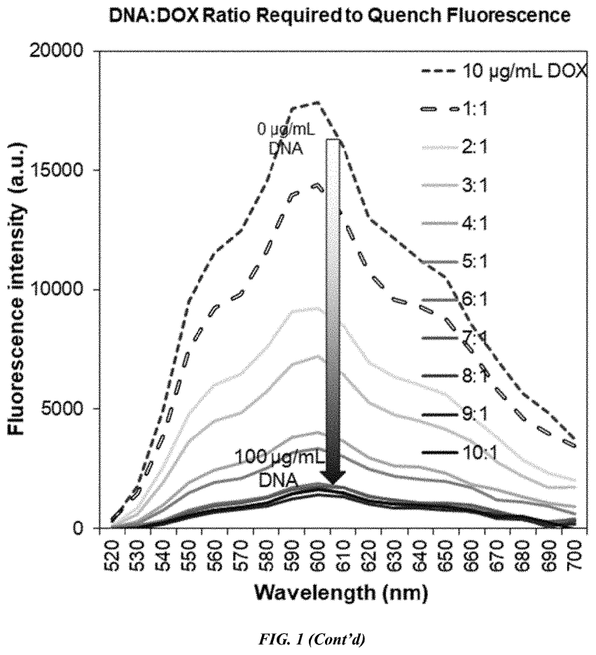

Physical Characterization of DOX/DNA complexes. Loading capacity and efficiency of DOX with DNA: A quenching study of DOX was performed using DNA to assess the loading capacity and the encapsulation efficiency of the DOX/DNA nanoparticle (see FIG. 1). The loading capacity and encapsulation efficiency of DOX/DNA was determined to be ˜14% and ˜88%, respectively. Fluorescence spectra of DNA:DOX ratios between 1-100 were first measured (FIG. 1, Top Panel), then fluorescence spectra of DNA:DOX ratios between 1-10 were measured (FIG. 1, Bottom panel) using an excitation at 490 nm. The most favorable weight ratio was determined from this was 6:1 DNA to DOX.

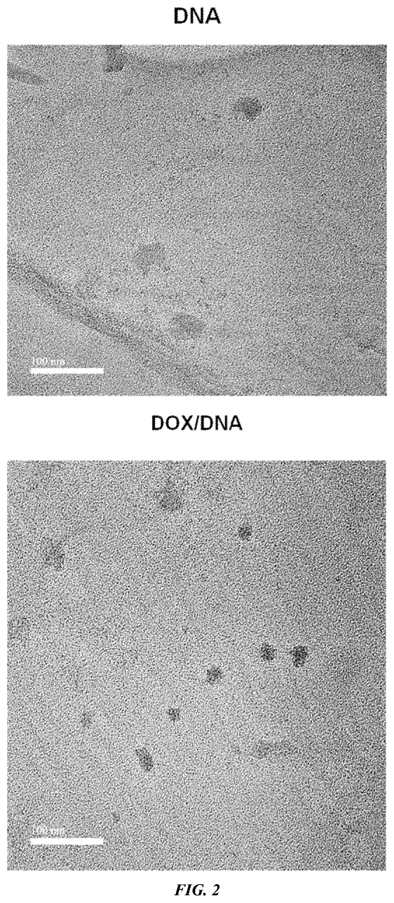

Size characterization of DOX/DNA complexes. Transmission electron microscopy was used to assess the size and morphology of DOX/DNA. DOX/DNA nanoparticles were prepared as described before diluting to a concentration of 1 μg/mL (DOX equivalent) in water (see FIG. 2) or PBS (see FIG. 3). The solution was allowed to rest for a further 30 minutes before being dropped onto a carbon grid for TEM imaging. TEM of DOX/DNA indicated a nanoparticle size of approximately 70 nm. These characterization studies indicated that the particles can carry a chemotherapeutic such as DOX, and the size characterization in particular demonstrated the ability of these particles to reach cancer cells.

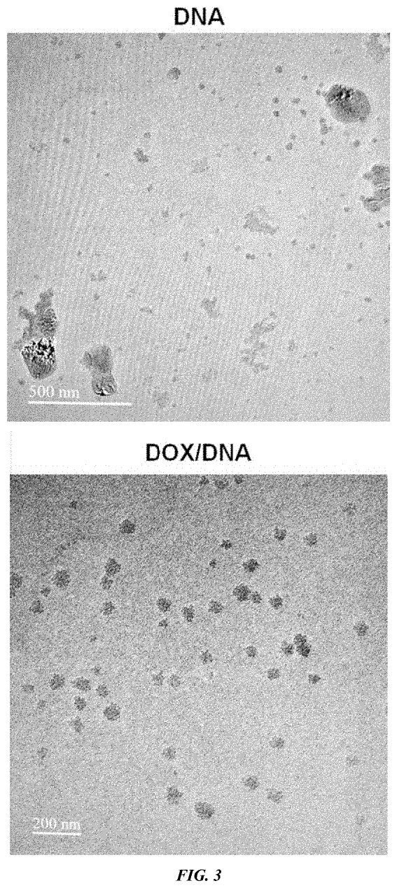

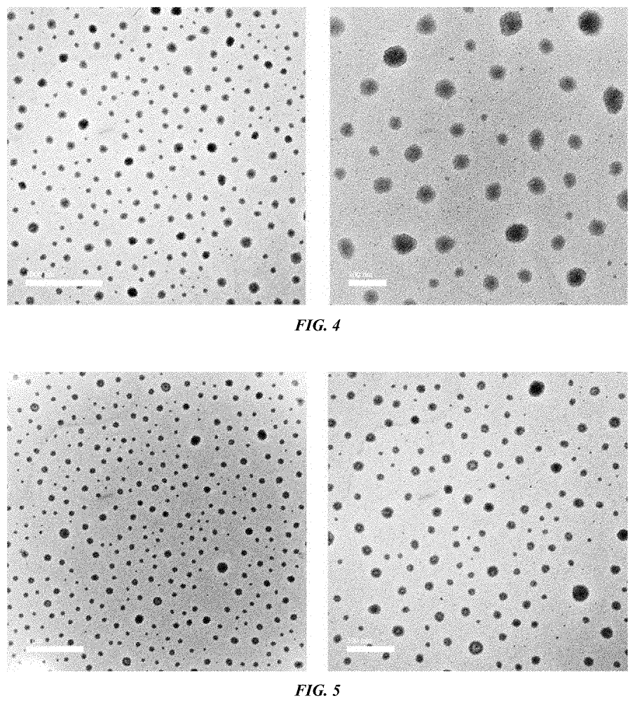

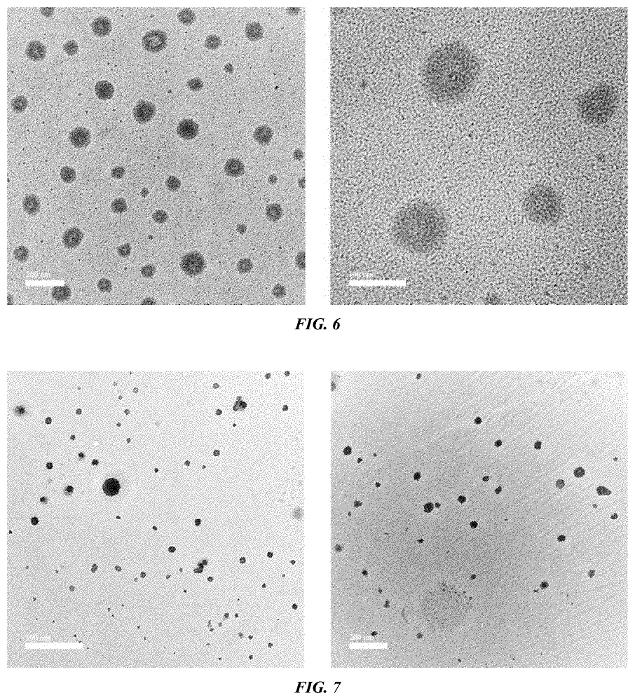

Long-term storage potential of DOX/DNA nanoparticles. Briefly, DOX/DNA was prepared in PBS, diluted with H₂O, lyophilized overnight, then reconstituted with H₂O and subsequently imaged. Final [DNA]=6 μg/mL and final [DOX]=1 μg/mL. The stability of DOX/DNA was largely unaffected by the lyophilization process (see FIGS. 4-6).

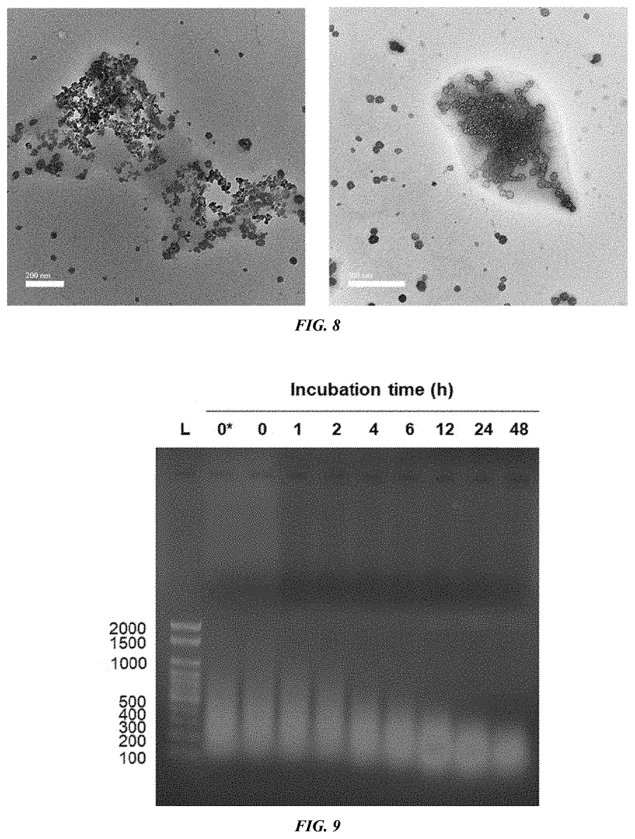

Stability of DNA in water and PBS. DNA was prepared in PBS and diluted in H₂O and then imaged. Final [DNA]=6 μg/mL. DNA remained largely stable in water or PBS (see FIGS. 7-8).

DNA Degradation in 10% FBS/PBS. To determine the stability of the DNA to serum exposure, DNA (100 μg/mL) was incubated with serum-containing PBS at 37° C. over a time period of 0 to 48 h. Samples were stored in −20° C. to stop enzymatic degradation from nucleases at each time point. DNA degraded serum in a time dependent manner (see FIG. 9). It is likely that the nucleases in the serum-containing media are contributing to the degradation of DNA. It is possible to infer a delayed release of DOX due to this degradation of DNA overtime in 10% FBS.

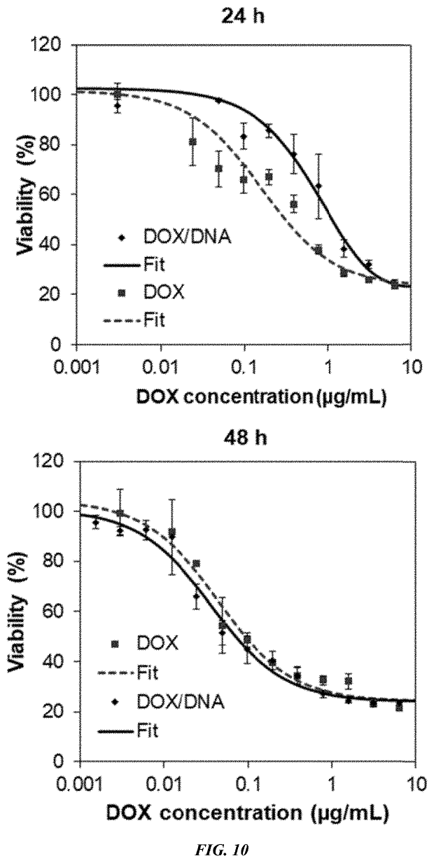

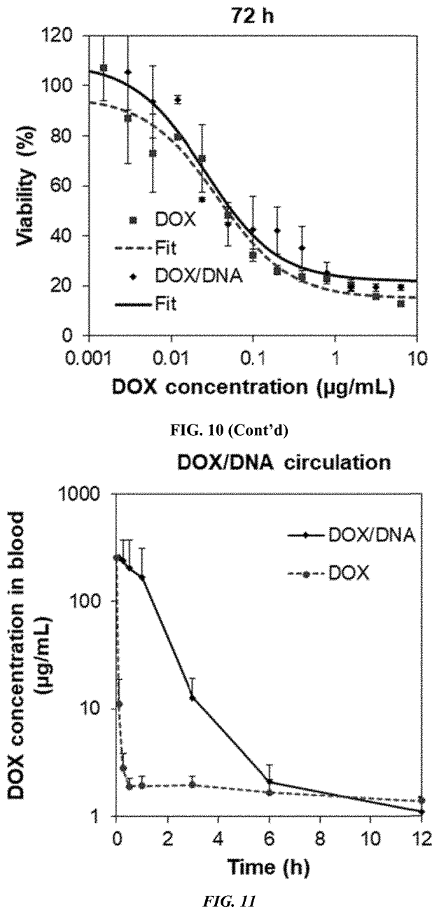

Cytotoxicity of DOX/DNA complexes in vitro at 24 h, 48 h, and 72 h. A study looking at in vitro cytotoxicity of DOX/DNA in EL4 cells at 24 h (left curve), 48 h (middle curve) and at 72 h (right curve) was performed. EL4 cells were treated in triplicate for 24, 48, and 72 hours with a range of concentrations, followed by cellular viability analysis via an MTT assay. DOX/DNA exhibited less cytotoxicity on these cells than DOX by a ˜3.5-fold difference at 24 h of incubation, as seen in the reported IC₅₀ values: DOX/DNA IC₅₀=1.143 μg/mL or 2.1 μM and DOX IC₅₀=0.313 μg/mL or 0.576 μM (see FIG. 10). DOX/DNA exhibited similar cytotoxicity on these cells compared to DOX at 48 and 72 h of incubation, as seen in the reported IC₅₀ values: DOX/DNA IC₅₀=0.072 μg/mL and DOX IC₅₀=0.093 μg/mL at 48 h or DOX/DNA IC₅₀=0.055 μg/mL and DOX IC₅₀=0.048 μg/mL at 72 h. This result in tandem with the 24 h cytotoxicity data suggests a delayed release of DOX from DOX/DNA. Moreover, the results demonstrate that the nanoparticles exhibit less toxicity in comparison to their free small molecule counterpart.

Pharmacokinetics of DOX/DNA in vivo. The results are shown of pharmacokinetics study performed on EL4-challenged C57BL/6 mice treated i.v with either 20 mg/kg DOX or 20 mg/kg DOX equiv. of DOX/DNA (see FIG. 11). The mice were 6-8-week-old female mice. It was found that DOX/DNA has a longer blood circulation residence half-life (DOX/DNA: T_{1/2}=75 minutes) in EL4-challenged C57BL/6 mice compared to mice treated with DOX (DOX: T_{1/2}=3 minutes), n=3. DOX is absorbed by the tissue in ˜15 minutes (as indicated by the steep initial slope of the curve), then a profile more reminiscent of hepatic and renal clearance was seen. DOX/DNA, however, exhibits a far less steep tissue absorption profile that endures for 1 hour. It can be inferred from the foregoing results, that there was enhanced circulation of DOX and DOX protection/shielding due to DNA. After which, a profile indicative of hepatic and renal clearance was observed. Accordingly, the drug delivery system of the disclosure alters the dissolution and absorption of doxorubicin, possibly allowing for sustained release of the active agent.

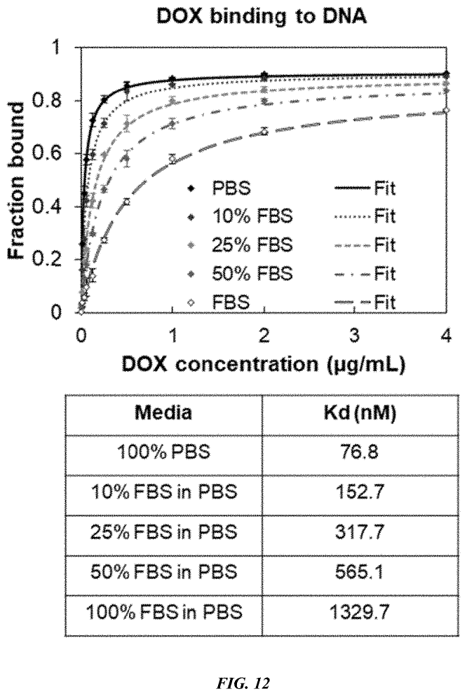

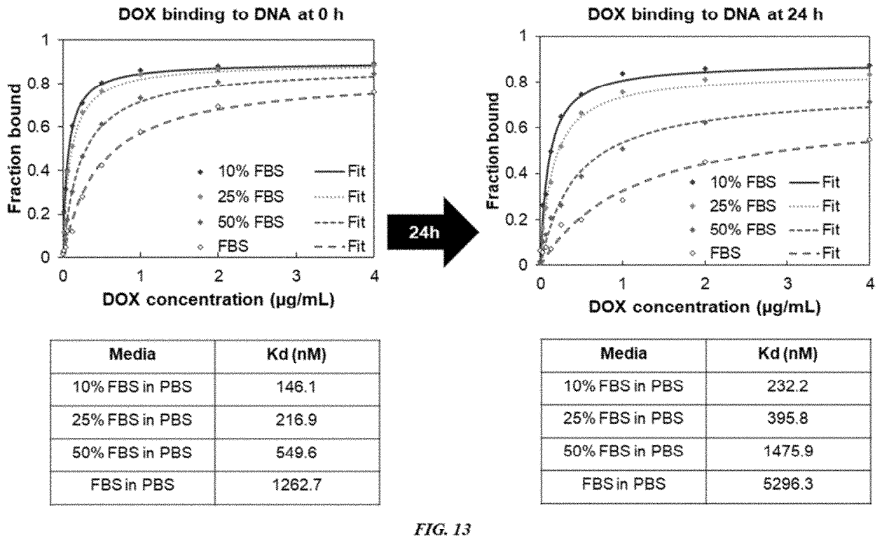

DOX/DNA disassociation kinetics in vitro. Binding kinetics of DOX with DNA was measured by observing fluorescence of DOX/DNA based on the concentration of DOX. Fluorescence of DOX/DNA was measured as [DOX] was increased. [DNA] remained constant at 400 μg/mL. Fluorescence was measured in using the Multi-Mode reader. Binding kinetics of DOX with DOX/DNA was studied in PBS, serum-containing PBS, or FBS. DOX dissociation from DOX/DNA increases with an increase in serum content and with an increase in time (see FIGS. 12-13). This data corroborates the data from the DNA degradation assay. K_{d} values for DOX dissociation from DOX/DNA in PBS, 10% FBS, 25% FBS, 50% FBS, and FBS were calculated to be 76.8 nM, 152.7 nM, 317.7 nM, 565.1 nM, and 1329.7 nM, respectively. This experiment clearly indicates that DOX is releasing from DNA due to FBS.

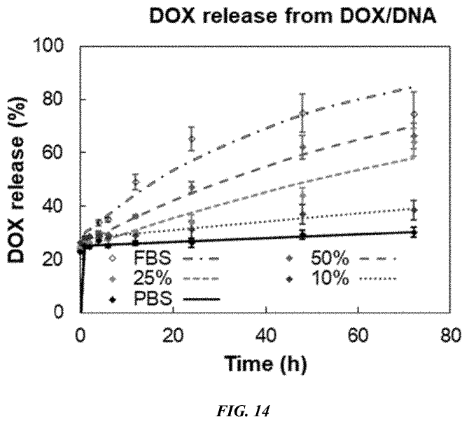

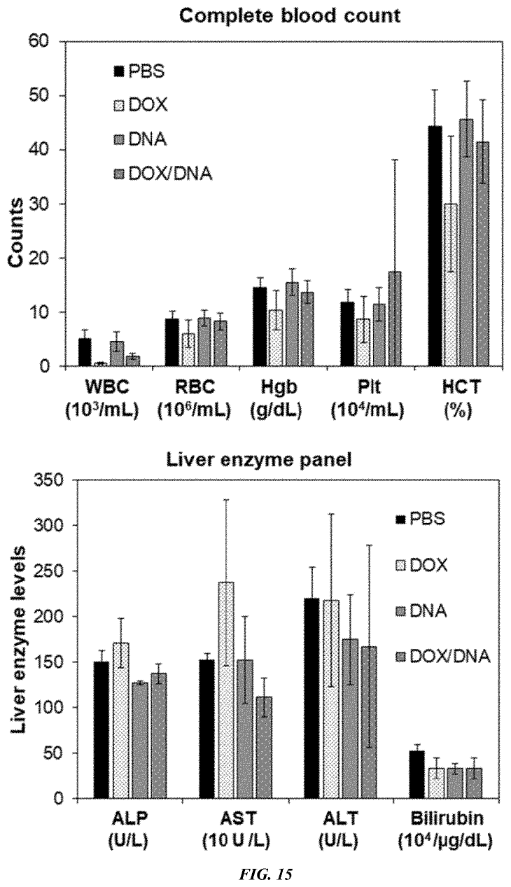

DOX release studies from DOX/DNA. Cumulative DOX release from DOX/DNA was performed in 100% PBS, 10% FBS/PBS, 25% FBS/PBS, 50% FBS/PBS, or 100% FBS over 72 hours. The highest DOX release from DOX/DNA was found when FBS was used (see FIG. 14). This data in tandem with the binding kinetics experiment suggests that DOX is released from DOX/DNA over time depending on the amount of serum content in the media. The majority of DOX should be released from the nanoparticles over 72 hours, at least according to this model.

Complete blood count and liver enzyme panel of DOX/DNA and DOX. A complete blood count and liver enzyme panel were conducted in C57BL/6 mice treated with 20 mg/kg DOX, 20 mg/kg DOX equiv. of DOX/DNA, PBS, or 120 mg/kg DNA, n=3. The mice used in this study were 6-12-week-old female C57BL/6/027 mice (Charles River Laboratories, Wilmington, Mass., USA). The mice were administered treatment via tail vein injection. After 24 h p.i., complete blood count (CBC), and liver enzyme levels were measured. Blood was collected by saphenous vein collection, mixed with EDTA, and analyzed with a hematology analyzer for white blood cells (WBC), red blood cells (RBC), hemoglobin (Hgb), platelets (Plt) and hematocrit (HCT). For the liver enzyme panel, 24 h p.i. serum was isolated from blood and sent to IDEXX Laboratories, Inc. (Westbrook, Me.) for alkaline phosphatase (ALP), aspartate aminotransferase (AST), alanine aminotransferase (ALT), and total bilirubin analysis. DOX was shown to have a greater impact on circulating blood cells and liver enzymes in comparison to DOX/DNA and DOXIL. Based upon the panels, DOX/DNA had significantly different modulating effects on blood components and liver enzymes than use of DOX alone (see FIG. 15).

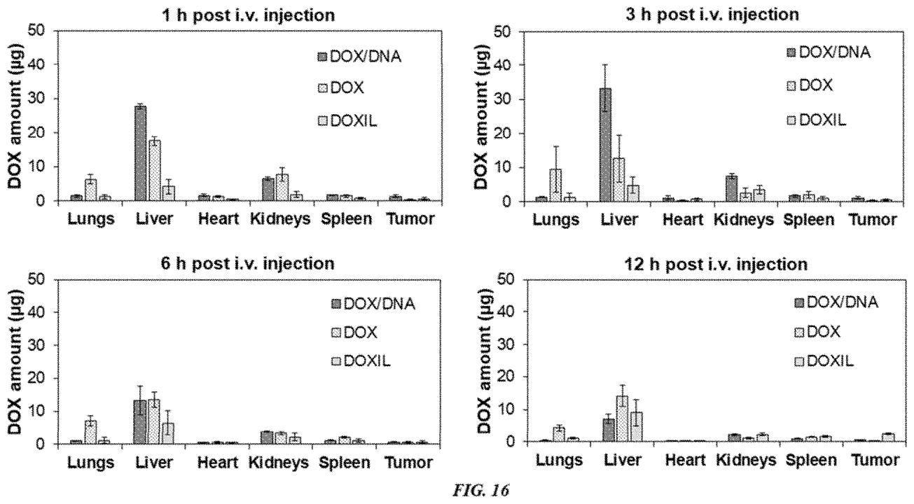

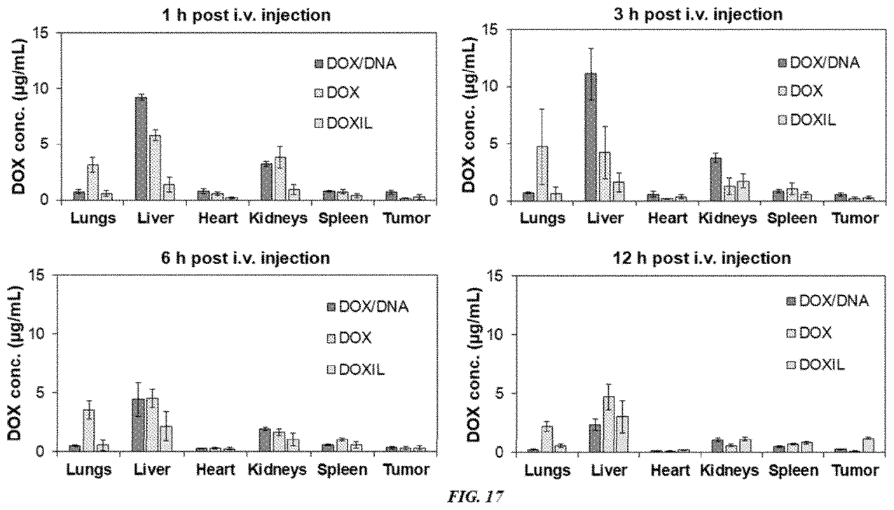

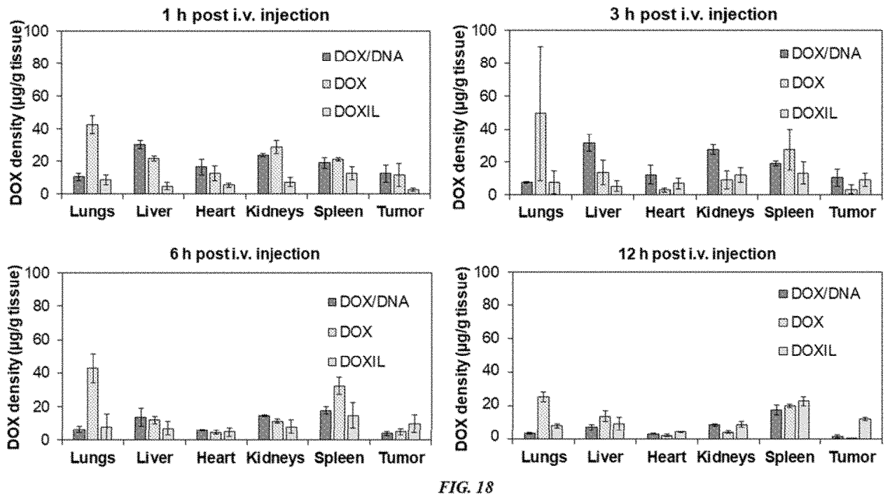

Biodistribution of DOX/DNA and DOX in vivo. DOX accumulation was characterized in organs and tumor tissue after a 20 mg/kg i.v. administration of DOX, DOXIL (20 mg/kg DOX equivalent), or DOX/DNA (20 mg/kg DOX equivalent) in EL4-challenged C57BL/6 mice (Female, 6-8 weeks old) at 1, 3, 6, and 12 hours. Accumulation of DOX in lungs is lower in DOX/DNA group, n=5 (except for DOXIL 12 h, where n=3) (see FIGS. 16-18). The greatest tumor accumulation of DOX was found in the mice treated with DOX/DNA. Accordingly, DOX/DNA improves drug delivery to the tumor site. Further, less organ toxicity was observed with DOX/DNA, specifically in the lungs and spleen. This is also highlighted by the greater levels of DOX cleared by the liver and kidneys. Larger particles, such as DOX/DNA, allow for macrophage uptake and are cleared from the lung, thus leading to less lung toxicity of DOX when delivered as DOX/DNA.

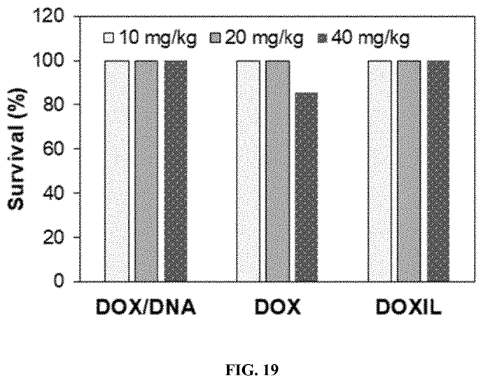

Acute toxicity survival curves in C57BL/6 mice. Acute toxicity was not observed in dose regimens 20 mg/kg or below, n=7. DOX-treated mice experience acute toxicity (cardiac arrest) due to 40 mg/kg dose administration (see FIG. 19). Accordingly, DOX/DNA is safer than DOX. DOX/DNA has a greater therapeutic window than DOX. In regards to DOXIL, DOX/DNA has more facile assembly process compared to DOXIL and can be produced more efficiently.

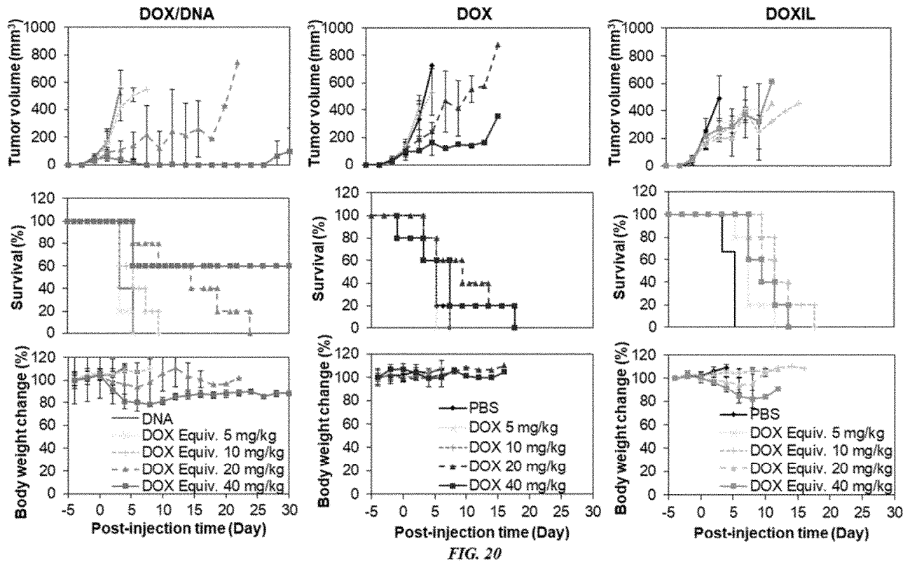

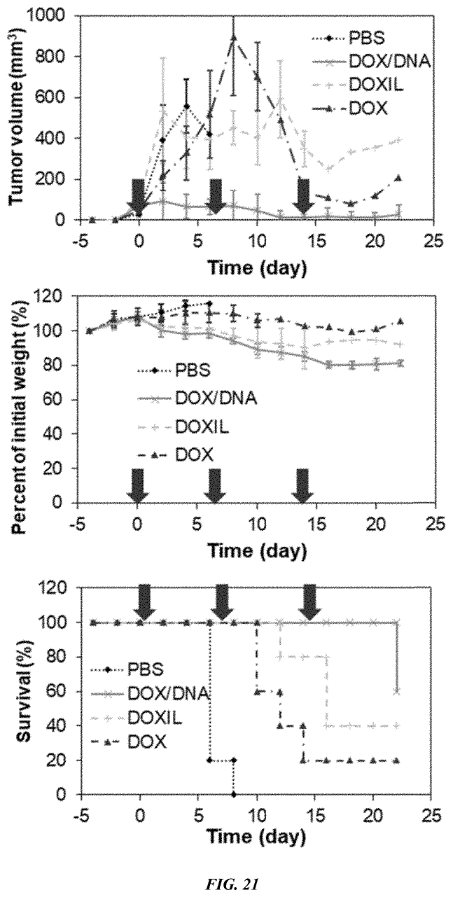

Efficacy of DOX, DOX/DNA, and DOXIL treatment on Tumor Growth and rate of survival in an EL4-cancer model. To validate the safety and efficacy of this drug delivery system, the tumor growth and survival of EL4-challenged mice were regularly tracked for 30 days after i.v treatment of DOX or DOX/DNA with a range of doses (2-3-month-old female mice). DOX/DNA slows tumor growth and improves survival rate in EL4-challenged C57BL/6 mice, n=5, better than DOX treatment alone (see FIG. 20)′ The 20 mg/kg dosage exhibited prolonged survival and slowed tumor growth when using the nanocarrier formulation. Interestingly, complete tumor regression was observed until day 28 in the mice treated with 40 mg/kg of DOX/DNA. Moreover, 60% of these mice survived to the endpoint of the experiment. DOX/DNA treatment leads to weight loss in high dose treatment in EL4-challenged C57BL/6 mice, n=5 (see FIG. 21). These results effectively demonstrate DNA's ability to increase the maximum tolerated dose of DOX, in addition to demonstrating its protective effects against systemic toxicity. Moreover, DNA treatment alone was similar to that of PBS, highlighting the safety of the drug delivery vehicle in this murine solid tumor model.

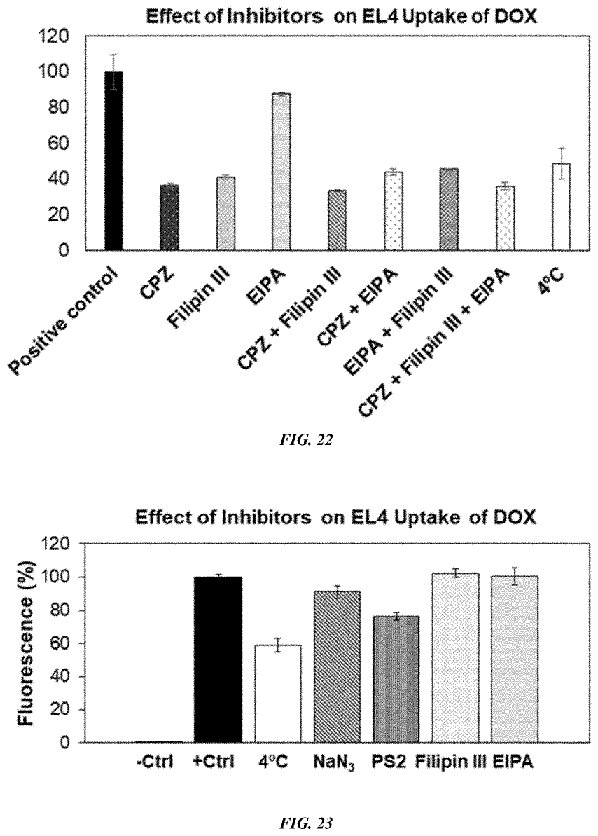

Endocytosis inhibition and the effect on DOX/DNA and DOX uptake. The effect of inhibitors CPZ (20 μM), Fillipin III (5 μM), EIPA (20 μM), and various combinations thereof, or 4° C. on EL4 cellular uptake was evaluated for DOX/DNA (see FIG. 22). Chlorpromazine (CPZ) is a clathrin-dependent pathway inhibitor. While, Filipin III is a caveolin-dependent pathway inhibitor. EIPA is a macropinocytosis pathway inhibitor. The concentrations chosen for the assay were determined using a dose-response assay for each inhibitor. It was found that DOX/DNA was taken up by the cells via clathrin-dependent and caveolin-dependent pathways. Membrane fusion was also involved, as indicated by 4° C. inhibition of DOX/DNA uptake.

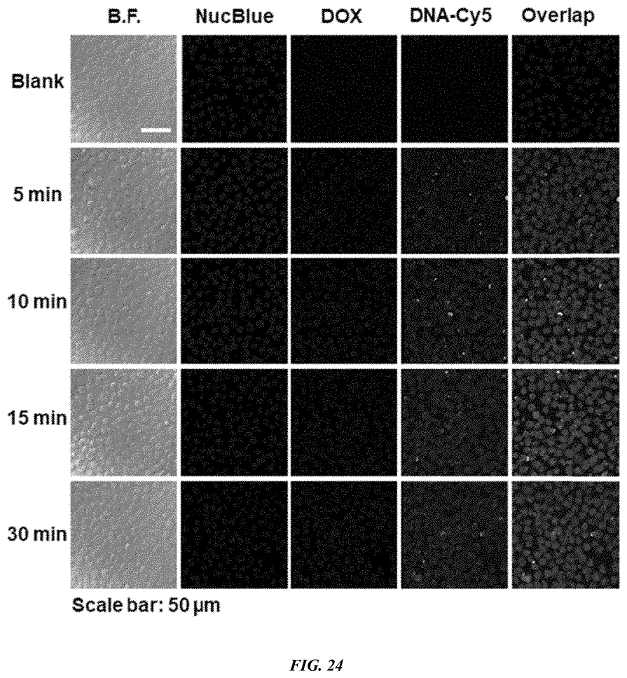

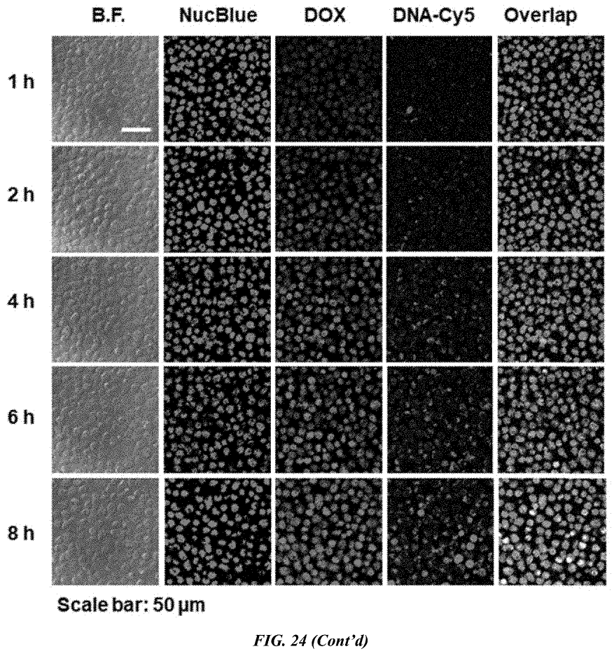

DOX uptake by EL4 cells was tested with inhibitors: NaN₃ (120 mM), PS2 (12 μg/mL), Filipin III (5 μg/mL), EIPA (20 μM), and 4° C. Uptake was measured using flow cytometry. Briefly, cells were primed for 15 minutes with the inhibitors before being treated for 1 hour with DOX. Cells containing DOX were counted with the flow cytometer using yellow fluorescence. In contrast to DOX/DNA, the inhibition studies suggest DOX uptake is primarily via membrane fusion (see FIG. 23).

Confocal Imaging of EL4 Cells Treated with DOX/DNA show localization of the treatment in the EL4 Cells. The CLSM images show that DOX/DNA was taken up by EL4 cells over time (see FIG. 24). The CLSM images also suggest internalization of the nanoparticle, and not just DOX alone.

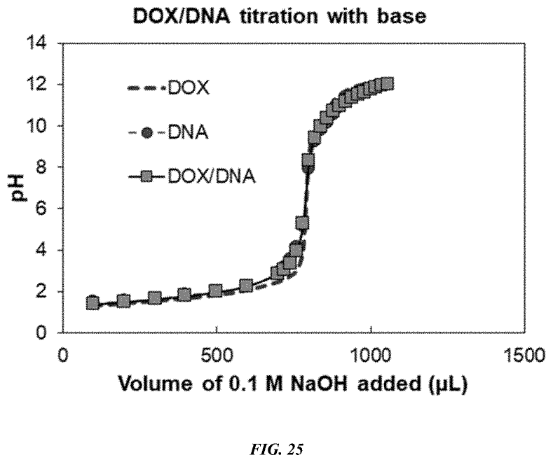

Titration of DOX, DNA, and DOX/DNA using a weak base. DOX, DNA or DOX/DNA were made in water at an initial volume of 1 mL. DOX equiv. at 600 μg/mL. The pH was then brought down below 2 with 1 M HCl, then the pH was adjusted higher using small volumes (100 μL or 20 μL) of 0.1 M NaOH. DOX, DNA and DOX/DNA all exhibited similar titration curves (see FIG. 25).

A number of embodiments have been described herein. Nevertheless, it will be understood that various modifications may be made without departing from the spirit and scope of this disclosure. Accordingly, other embodiments are within the scope of the following claims.

1. A composition comprising one or more therapeutic compounds that are complexed with nucleic acid fragments to form nanoparticles, wherein the one or more therapeutic compounds are small molecules that can associate or bind with DNA or RNA, wherein the nucleic acid fragments are complexed with the one or more therapeutic compounds at a wt/wt ratio of 2:1 to 10:1.

2. (canceled)

3. The composition of claim 1, wherein the nucleic acid fragments are complexed with the one or more therapeutic compounds at a wt/wt ratio of 4:1 to 7:1.

4. (canceled)

5. The composition of claim 1, wherein the nanoparticles are from 10 nm to 500 nm in size.

6. (canceled)

7. The composition of claim 1, wherein the one or more therapeutic compounds comprises anthracyclines, anthracenediones, camptotheca compounds, podophyllum compounds, minor groove binders, bleomycin, and/or actinomycin D.

8. The composition of claim 1, wherein the one or more therapeutic compounds comprises aclarubicin, doxorubicin, daunorubicin, idarubicin, epirubicin, amrubicin, pirarubicin, valrubicin, and/or zorubicin.

9. The composition of claim 8, wherein the one or more therapeutic compounds comprises doxorubicin.

10. The composition of claim 1, wherein the one or more therapeutic compounds comprises mitoxantrone, topetecan, etoposide, teniposide, bleomycin, actinomycin D, and/or duocarmycin A.

11. The composition of claim 1, wherein the one or more of the nucleic acid fragments comprise a ligand that targets the nanoparticles to specific cells, tissue, organs, or tumors.

12. The composition of claim 1, wherein the nucleic acid fragments comprise fragments of naturally occurring DNA, RNA and/or DNA-RNA hybrids.

13. The composition of claim 1, wherein the nucleic acid fragments comprise chemically synthesized DNA, RNA and/or DNA-RNA hybrids of differing nucleotide lengths.

14. The composition of claim 13, wherein the RNA has been modified to replace the 2′ ribose hydroxyl group with an —O-alkyl group or a halide.

15. The composition of claim 1, wherein the nucleic acid fragments are DNA fragments.

16. The composition of claim 1, wherein the nucleic acid fragments are from salmon DNA.

17. The composition of claim 1, wherein the nucleic acid fragments are from 20 nt to 10,000 nt in length.

18. The composition of claim 17, wherein the nucleic acid fragments are from 50 nt to 2,000 nt in length.

19-25. (canceled)

26. A method of treating a human subject having a cancer in need of treatment thereof, comprising: administering an effective amount of the composition of claim 1 to the subject.

27. The method of claim 26, wherein the cancer is selected from acute lymphoblastic leukemia, acute myeloblastic leukemia, bone sarcoma, breast cancer, endometrial cancer, gastric cancer, head and neck cancer, Hodgkin lymphoma, Non-Hodgkin lymphoma, liver cancer, kidney cancer, multiple myeloma, neuroblastoma, ovarian cancer, small cell lung cancer, soft tissue sarcoma, thyomas, thyroid cancer, transitional cell bladder cancer, uterine sarcoma, Wilms' tumor, and Waldenström macroglobulinemia.

28. The method of claim 26, wherein the method further comprises administering to the subject with one or more anticancer agents selected from angiogenesis inhibitors, tyrosine kinase inhibitors, PARP inhibitors, alkylating agents, vinca alkaloids, anthracyclines, antitumor antibiotics, antimetabolites, topoisomerase inhibitors, aromatase inhibitors, mTor inhibitors, retinoids, and HDAC inhibitors.

29. The method of claim 28, wherein the method further comprises administering to the subject with one or more anticancer agents selected from mitoxantrone, topetecan, etoposide, teniposide, bleomycin, actinomycin D, and duocarmycin A.

30. The composition of claim 11, wherein the ligand binds to a tumor specific antigen.
